(19) United States
(12) Patent Application Publication
(10) Pub. No.: US 2013/0109738 A1
(43) Pub. Date: May 2, 2013

(54) Control of Cardiac Growth, Differentiation and Hypertrophy

(71) Applicants: Ching-Pin Chang, Cupertino, CA (US); Calvin Hang, Stanford, CA (US); Pei Han, Mountain View, CA (US); Jin Yang, Mountain View, CA (US); Ksiu-Ling Cheng, Taipei (TW)

(72) Inventors: Ching-Pin Chang, Cupertino, CA (US); Calvin Hang, Stanford, CA (US); Pei Han, Mountain View, CA (US); Jin Yang, Mountain View, CA (US); Ksiu-Ling Cheng, Taipei (TW)

(73) Assignee: The Board of Trustees of the Leland Stanford Junior University, Palo Alto, CA (US)

(21) Appl. No.: 13/580,122

(22) PCT Filed: Feb. 23, 2011
(86) PCT No.: PCT/US2011/025945

§ 371 Date: Jan. 10, 2013

Related U.S. Application Data
(60) Provisional application No. 61/338,899, filed on Feb. 24, 2010.

(51) Int. Cl. A61K 31/7088; A61K 31/138; G01N 33/68

(52) U.S. Cl. CPC A61K 31/7088; G01N 33/6887; A61K 31/138
USPC 514/44 A; 435/7.1; 800/3; 800/9; 435/375; 514/651

(57) ABSTRACT

Methods and compositions are provided for the diagnosis and treatment of heart diseases relating to cardiac hypertrophy, and for the regulation of proliferation and differentiation of cardiomyocyte progenitors in vitro. The detection of expression of components of the BAF complex, including, without limitation, detection of expression of Brg1, provides useful methods for early detection, diagnosis, staging, and monitoring of conditions leading to hypertrophy and enlargement of the heart. Manipulation of Brg1 activity provides for therapeutic intervention in the development of cardiac hypertrophy, where methods of decreasing Brg1 activity, e.g. through inhibition of binding, decreasing expression, and the like, reduces cardiac hypertrophy.

Cardiac hypertrophy is recognized as one of the independent risk factors leading to severe heart diseases such as ischemic heart diseases and heart failure. The Framingham Heart Study demonstrated that when cardiac hypertrophy is present, there is a 2.5 to 3 fold increase in the percentage of onset of heart failure, ischemic heart diseases such as angina pectoris and myocardial infarction, and cardiovascular diseases such as arrhythmia. Cardiac hypertrophy is a maladaptive mechanism made in response to an increased workload imposed on the heart. It is a specialized process reflecting a quantitative increase in cell size and mass rather than cell number, and may be the result of one or a combination of stimuli.

Cardiomyocytes differentiate during embryogenesis. They maintain a capacity to divide in embryos even after differentiation, and actively increase by division in the fetal period, but their capacity for growth suddenly drops after birth. As a consequence, subsequent growth of the heart occurs primarily by physiological enlargement, specifically, by increasing the size of the individual cardiomyocytes. Cardiac hypertrophy is caused either by an increase of the width of myofibrils or by an increase of the length of myofibrils. These contrasting hypertrophic forms are derived respectively by parallel assembly and serial assembly of the sarcomeres, and termed concentric and eccentric hypertrophy, respectively.

Cardiac hypertrophy can be induced by response to normal post-natal physiological adaptation or by movement, resulting in increased cardiac pump capacity corresponding to the increase in demand. However, a pathologically generated load on the heart may also induce cardiac hypertrophy that leads to heart disease. When the load on the ventricles is increased by hypertension or valvular disease of the heart, or when damage to the cardiomyocytes themselves is produced by myocardial infarction or myocarditis, pathological cardiac hypertrophy can occur. Cardiac hypertrophy is a compensatory mechanism of the heart to adapt to the increased mechanical load. However, prolonged cardiac hypertrophy results in systolic and diastolic dysfunctions of the heart, and eventually heart failure. Also, hypertrophic hearts become susceptible to ischemic heart disease and prone to fatal arrhythmia.

During the attempts to identify and stabilize the underlying causes of cardiomyopathy, treatment is usually instituted to minimize the symptoms and optimize the efficiency of the failing heart. Medication remains the mainstay of treatment for heart failure. Heart failure refractory to medication requires transplant surgery. Dilated cardiomyopathy (or eccentric cardiac hypertrophy) has been indicated as the most common cause for cardiac transplantation in the U.S.

Conventional pharmacologic methods to treat chronic heart failure relied on inotropic drugs, with the objective of improving systolic capacity of the heart and to increase the cardiac output. Although inotropic drugs improved subjective symptoms and exercise tolerance, they failed to prolong life. In fact, these inotropic agents increase mortality. Newer therapies include inhibitors of angiotensin conversion enzyme (ACE), which suppresses the onset and development of cardiac hypertrophy in animal models, endothelin antagonists and vasopressin antagonists.

Non-pharmacological treatment is primarily used as an adjunct to pharmacological treatment. One means of non-pharmacological treatment involves reducing the sodium in the diet. In addition, non-pharmacological treatment may include the elimination of precipitating drugs, including negative inotropic agents, cardiotoxins and plasma volume expanders.

The treatment of cardiac hypertrophy is of great interest. As evidenced by the present invention, underlying mechanisms of hypertrophy relate to fundamental aspect of cardiomyocyte differentiation and control of gene expression directly relevant to the clinical outcome of heart disease.

The execution of transcriptional programs during development relies on precise temporal- and spatial-specific regulation of gene expression, which in turn requires the modulation of chromatin structure of target genes. An important class of enzymes capable of manipulating chromatin structure is the “chromatin-remodeling complexes,” the multisubunit molecular motors that use energy derived from ATP hydrolysis to physically change histone-DNA contacts. A best-known chromatin remodeler in mammals is the Brg1-associated factor (BAF) complex related to the yeast Swi-Snf complex. The BAF complex contains ˜12 subunits, including the ATPase Brg1 or its homologue Brm. In addition to Brg1/Brm, several other subunits of the BAF complex are encoded by gene families, thus leading to the combinatorial assembly and generation of perhaps hundreds of complexes with divergent functions.

Remodeling of chromatin can lead to activation of gene expression in vitro. For example, the SWI/SNF chromatin remodeling complex can potentiate transcriptional activity. There are also several examples of a requirement for the activity of chromatin remodeling complexes for gene activation in vivo. The SWI/SNF chromatin remodeling complex is required for the activity of the glucocorticoid receptor, and for activation of the hsp70 gene. Mutations in the yeast SWI/SNF gene result in a decrease in expression of one group of genes and an increase in expression of another group of genes, showing that chromatin remodeling can have both positive and negative effects on gene expression.

The present invention demonstrates a role for a chromatin remodeling complex in the differentiation and growth, including hypertrophic growth, of cardiomyocytes. The control of these factors provides a means of directing cardiac progenitor growth, and the diagnosis and treatment of cardiac hypertrophy and myopathy.

Publications relating to the invention may include Takeuchi et al. Nature. 2009 459(7247):708-11; “Directed transdifferentiation of mouse mesoderm to heart tissue by defined factors”; which proposes a role for BAF in the activation of cardiac differentiation. Gao et al. Proc Natl Acad Sci USA. 2008 105(18):6656-61, “ES cell pluripotency and germ-layer formation require the SWI/SNF chromatin remodeling component BAF250a”; and (WO 2008/088882) METHODS OF GENERATING CARDIOMYOCYTES.

### SUMMARY OF THE INVENTION

The present invention provides methods and compositions for the diagnosis and treatment of heart diseases relating to cardiac hypertrophy. The detection of expression of components of the BAF complex, including, without limitation, detection of expression of Brg1, provides useful methods for early detection, diagnosis, staging, and monitoring of conditions leading to hypertrophy, enlargement, and myopathy of the heart. It has been found that increased expression of Brg1 compared to normal tissue is indicative of cardiac hypertrophy in mammals, including human patients. Manipulation of Brg1 activity provides for therapeutic intervention in the development of cardiac hypertrophy, where methods of decreasing Brg1 activity in cardiac endothelial cells and/or cardiomyocytes, e.g. through inhibition of binding to cognate receptors, target genes, decreasing expression, and the like, reduces cardiac hypertrophy and myopathy.

The invention also provides methods for the identification of compounds that modulate cardiac hypertrophy, e.g. through specific inhibitions of Brg1 activity and/or expression, as well as methods for the treatment of disease by administering such compounds to individuals exhibiting heart failure symptoms or tendencies.

The invention also provides means of manipulating cardiomyocyte progenitor differentiation in vitro through manipulation of expression and activity of proteins in the BAF complex, including without limitation, Brg1. Expression of Brg1 is shown to suppress myocyte differentiation, to control myosin heavy chain expression, and to regulate proliferation.

### BRIEF DESCRIPTION OF THE DRAWINGS

FIG. 1. Embryos lacking myocardial Brg1 die at E11.5-E12.5 (A) Whole mount β-galactosidase staining of Sm22αCr;R26R embryos. (B and C) Immunostaining of Brg1 (red) and Troponin T (green) of E9.5 control and Sm22αCre;Brg1F/F embryos. (D and E) Control and mutant (Sm22_Cre;Brg1F/F) embryos whole mount at E11.5. (F) The frequency of recovering mutant embryos at different gestational dates.

FIG. 2. Myocardial Brg1 maintains BMP10 expression to promote myocardial proliferation (A-F) H&E sections of E10.5 (A-D) and E11.5 (E, F) embryos. Sm22αCre;Brg1^{F/F} embryos display thin myocardium (arrowheads) and no interventricular septum (arrows). Asterisks: endocardial cushion. (C and D) are magnification of brackets in (A) and (B), respectively. (G) Myocardial thickness quantitation at E10.5. p-value: Student-t test. (H) Trabecular quantification at E10.5. (I-L) BrdU immunostaining (brown) of E10.5 embryos. (I, J): ventricular wall. (K, L): septal primordia. (M) Myocardial BrdU incorporation quantitation. (N and O) RNA in situ hybridization of BMP10 (brown) in the myocardium at E10.5. (P and Q) Immunostaining of p57kip2 (brown) of E10.5 hearts.

FIG. 3. BMP10 rescues myocardial proliferation defects in Sm22αCre;Brg1^{F/F} embryos (A-D) Gross morphology of cultured embryos. (E-H) BrdU immunostaining (brown) of cultured embryos treated with BrdU during the last six hours. Arrows: myocardium, arrowheads: endocardium. (I) Myocardial BrdU incorporation quantitation of cultured embryos. p-value: Student-t test.

FIG. 4. Brg1 suppresses myocardial differentiation and controls MHC expression (A and B) α-actinin immunostaining (green) of E9.5 embryos. Nuclei: blue. Arrowheads: trabecular myocardium. Arrows: compact myocardium. (C and D) Transmission electron micrographs of the compact myocardium of E10.5 embryos. Arrows: sarcomeres. Arrowheads: Z-lines. (E) Quantitative RT-PCR analysis of ventricular α-MHC and β-MHC expression of embryos at E10.5 and E11.5. Ctrl: control embryos. Mut: Sm22αCre;Brg1^{F/F}embryos. p-value: Student-t test. (F) Sequence alignment of the α-MHC locus from mouse, human, and rat. Height of individual peaks indicates degree of sequence homology. Black boxes (a1-a7) are regions of high sequence homology and further analyzed by chromatin immunoprecipitation. Red: promoter elements. Salmon: introns. Yellow: untranslated regions. Blue: exons. (G) PCR analysis of Brg1-immunoprecipitated chromatin from E11.5 hearts. (a1-a7) indicate conserved regions of α-MHC promoter. (H) Luciferase reporter assays of α-MHC promoter activity in SW13 cells. (I) Immunostaining of HDAC1, 2 and 3 (brown) in E11.5 hearts. (J) Co-immunoprecipitation of Brg1 and HDAC1 and HDAC2 proteins in E11.5 hearts. (K) Sequence alignment of the β-MHC locus from mouse, human, and rat. Black boxes (b1-b5) are regions of high sequence homology and further analyzed by chromatin immunoprecipitation. Green: transposons and simple repeats. (L) PCR analysis of Brg1-immunoprecipitated chromatin from E11.5 hearts. (b1-b5) indicate conserved regions of β-MHC promoter. (M) Luciferase reporter assays of β-MHC promoter activity in SW13 cells.

FIG. 5. Brg1 commands parallel pathways to regulate myocardial proliferation and differentiation (A and B) Gross morphology of embryos cultured from E9.5 for 24 hours. (C and D) Quantitative RT-PCR analysis of α-MHC and β-MHC mRNA (C) and α-MHC/β-MHC ratio (D) in cardiac ventricles of cultured embryos treated with DMSO or TSA. p-value: Student-t test. (E and F) Quantitative RT-PCR analysis of ventricular α-MHC and β-MHC mRNA (E) and α-MHC/β-MHC ratio (F) in cultured embryos treated with BSA or BMP10. Ctrl: control embryos. Mut: Sm22αCre;Brg1^{F/F} embryos. (G and H) BrdU immunostaining (brown) of cultured embryos treated with DMSO (G) and TSA (H). (I) BrdU quantitation in myocardial cells of cultured embryos (G, H).

FIG. 6. Brg1 is required for cardiac hypertrophy, fibrosis and MHC switches in adult mice (A) Section of a whole mount β-galactosidase stained (blue) heart of Tnnt2-rtTA;Tre-Cre; Rosa26LacZ mice. Red: nuclei counterstain. (B, C, D, E) Wheat agglutinin immunostaining (green) outlines cardiomyocyte cell border of papillary muscles at the mid-cavitary level of the left ventricle. Control and Tnnt2-rtTA;Tre-Cre;Brg1^{F/F} mice were sham-operated (B and D) or had received TAC (C and E) 4 weeks before. (F) Cardiomyocyte size quantitation, Ctrl: control mice. Mut: Tnnt2-rtTA;Tre-Cre; Brg1^{F/F} mice. p-value: Student-t test (G) Cardiac ventricular weight/body weight ratio. (H, I, J, K) Trichrome staining of control (H, J) and doxycycline-treated Tnnt2-rtTA;Tre-Cre;Brg1F/Fmice (I, K) 4-8 weeks after the TAC procedure. Black: nuclei, red: cardiomyocytes, blue: fibrotic areas. (L, M) Quantitative RT-PCR analysis of relative α-MHC and β-MHC changes in cardiac ventricles of doxycycline-treated control and Tnnt2-rtTA;Tre-Cre; Brg1^{F/F} (mutant) mice 4 weeks after the sham or TAC procedure. (N) Brg1 immunostaining (brown) in ventricular myocardium of control and Tnnt2-rtTA;Tre-Cre;Brg1′^{F} mice treated doxycycline and undergone sham or TAC procedure. (O) Quantitative RT-PCR analysis of Brg1 mRNA in wildtype mice 2 weeks after the sham or TAC procedure. (P) Immunoblot of Brg1 of whole heart nuclear extracts from TAC (for two weeks) and sham operated wild-type mice, normalized to Histone H1. (Q) PCR analysis of Brg1- and PARP1-immunoprecipitated chromatin from adult hearts from mice that had undergone TAC. Numbers (a1-a7) and (b1-b5) indicate conserved regions of α-MHC and β-MHC promoter. (R and S) Luciferase reporter assays of α-MHC (R) and β-MHC (S) promoter activity in SW13 cells with PARP1 inhibition. (T) Co-immunoprecipitation of Brg1, HDAC2, and PARP1 in TAC-treated adult hearts and in E11.5 hearts. (U) RT-PCR analysis of α-MHC and β-MHC in SW13 cells treated with HDAC and PARP1 inhibitors.

FIG. 7. Brg1 expression is elevated in patients with hypertrophic cardiomyopathy (A) Demographic data of control subjects and patients with hypertrophic cardiomyopathy (HCM). (B) Cardiac MRI of a normal subject and a HCM patient listed in (A). The arrows denote the thickness of interventricular septum measured during diastole (IVSd). LV, RV: left, right ventricle. LA, RA: left, right atrium. (C) Myocardial thickness (IVSd), and α-MHC, β-MHC, and Brg1 expression in normal and HCM subjects. (D and E) Working model of BAF function during embryonic development and pathological remodeling of the heart.

FIG. 8. Brg1-null myocardium does not have increased cell death (A-D) TUNEL staining of E10.5 (A and B) and E11.5 (C and D) wildtype and Sm22αCre; Brg1^{F/F} embryos. Very few cells of both wildtype and mutants are TUNEL-positive.

FIG. 9. Proliferation defects of Brg1-null myocardium is cell-autonomous (A) Quantification of BrdU incorporation of endocardium, endocardial cushion, and epicardium of control and Sm22αCre; Brg1^{F/F} embryonic hearts.

FIG. 10. Sm22αCre; Brg1^{F/F} mutants have normal expression of many cardiac genes RNA in-situ hybridization of E11.5 control and Sm22αCre; Brg1F/F hearts. Expression of these transcripts is comparable in amount and localization between control and mutants.

FIG. 11. Sm22αCre;Brg1^{F/F} mutants have ectopic expression of p57kip2(A) Quantitation of wild-type BrdU incorporation and p57kip2 expression, which show inverse correlation with each other. (B) p57kip2 immunostaining of E10.5 control and Sm22αCre;Brg1^{F/F} hearts show that the mutant has ectopic expression of p57kip2 in the septal primordium.

FIG. 12. Regulation of BMP1O, p57kip2 and myocardial proliferation is a cell-autonomous function of Brg1 in the myocardium (A and B) Immunostaining of Brg1 in Mef2cCre;Brg1^{F/+} (A) and Mef2cCre;Brg1^{F/F} (B) embryonic hearts at E10.5, showing that Brg1 (green) is deleted only in the right ventricular myocardium of Mef2cCre;Brg1^{F/F} embryos. Nuclei are stained blue by Hoescht. Arrow: right ventricular myocardium. Asterisks: endocardium. Arrowheads: left ventricular myocardium. RV: right ventricle. LV: left ventricle. (C) RNA in situ hybridization of BMP10 in control Mef2cCre;Brg1^{F/+} embryo at E10.5. BMP/10 is expressed in both right (arrow) and left (arrowheads) ventricular myocardium. BMP10 RNA signals are blue, and nuclei are counterstained red. (D) RNA in situ hybridization of BMP10 in mutant Mef2cCre;Brg1^{F/F} embryos at E10.5. BMP10 expression is expressed normally in the left ventricle (arrowheads), but severely diminished in the right ventricular (RV) myocardium (arrow). (E) Immunostaining of p57kip2 in control Mef2cCre;Brg1^{F/+} embryos at E10.5. p57kip2 (brown) is detected in the endocardium (asterisks), but not in the right (arrow) or left (arrowhead) ventricular myocardium. Nuclei are counterstained blue with hematoxylin. (F) Immunostaining of p57kip2 in mutant Mef2cCre;Brg1^{F/F} embryos at E10.5. p57kip2 is detected in the endocardium (asterisks) and in the right ventricular myocardium (arrows). It remains absent in the left ventricular myocardium (arrowhead). (G) Immunostaining of BrdU in control E10.5 Mef2cCre;Brg1^{F/+} embryos labeled with BrdU for six hours. BrdU (brown) is incorporated in the endocardium (asterisks) as well as in the right (arrows) and left (arrowheads) ventricular myocardium. Nuclei are counterstained blue with hematoxylin. H) Immunostaining of BrdU in mutant E10.5 Mef2cCre;Brg1^{F/F} embryos labeled with BrdU for six hours. BrdU is incorporated in the endocardium (asterisks) and left ventricular myocardium (arrowheads). BrdU incorporation is severely diminished in the right ventricular myocardium (arrows).

FIG. 13. PARP1 is expressed in embryonic myocardium and form complex with HDAC in adult myocardium. (A) PARP1 immunostaining of E11.5 embryonic heart. Asterisks denote myocardial cells. (B) Co-immunoprecipitation of PARP1 and HDAC2 from adult myocardium. (C) PARP inhibition by PJ-34 in embryos cultured from E9 to E10 causes MHC switch from β-MHC to α-MHC, (D) ChIP analysis of PARP proteins show that PARP1 binds to the promoters of both α- and β-MHC in E11.5 embryonic hearts.

FIG. 14a, b, Brg1 and Pecam1 co-staining in ventricular myocardium of wild type mice 2 weeks after sham/TAC operation. Brg1: red channel; Pecam1: green channel; Arrows: cardiac endothelial nuclei. c, d, β-galactosidase staining of SclCre^{ER};Rosa heart showing the presence of SclCre activity in the interstitial cells. Blue: X-gal staining. e, f, Brg1 and Pecam co-staining in ventricular myocardium of tamoxifen treated WT and SclCre^{ER};Brg1^{F/F} littermates 4 weeks after TAC operation. Brg1: red channel; Pecam: green channel; Arrows: cardiac endothelial nuclei.

FIG. 15. Cardiac endothelial Brg1 is required for cardiac hypertrophy, fibrosis in adult mice. a, Gross size of tamoxifen treated control and SclCre^{ER};Brg1^{F/F}littermate hearts after 4 weeks TAC operation. b, Heart to body weight ratio quantitation of control and SclCre^{ER};Brg1^{F/F} mice 4 weeks after sham/TAC operation. Ctrl: control. Mut: SclCre^{ER};Brg1^{F/F}. c-f, Wheat germ agglutinin (WGA) staining of tamoxifen-treated control and SclCre^{ER};Brg1^{F/F} hearts 4 weeks after sham/TAC operation. g, Cardiomyocyte size quantitation of control and SclCre^{ER};Brg1^{F/F} mice 4 weeks after sham/TAC operation. Ctrl: control. Mut: SclCre^{ER};Brg1^{F/F}. h-k, Trichrome staining of tamoxifen-treated control and SclCre^{ER};Brg1^{F/F} hearts 4 weeks after TAC. Black: nuclei, red: cardiomyocytes, blue: fibrosis.

### DETAILED DESCRIPTION OF THE EMBODIMENTS

Methods and compositions for the diagnosis and treatment of heart diseases including but not limited to cardiomyopathies; heart failure; and the like, are provided. The invention is based, in part, on the evaluation of the expression and role of genes that are differentially expressed in the heart, including cardiomyocytes and endothelial cells, in response to pressure overload. The detection of expression of components of the BAF complex, including, without limitation, detection of expression of Brg1, provides useful methods for early detection, diagnosis, staging, and monitoring of conditions leading to hypertrophy, enlargement, and myopathy of the heart. The invention also provides methods for the identification of compounds that modulate cardiac hypertrophy and myopathy, e.g. through specific inhibitions of Brg1 activity and/or expression, as well as methods for the treatment of disease by administering such compounds to individuals exhibiting heart failure symptoms or tendencies.

The present inventors studied the role of the BAF complex, including Brg1, in cardiac hypertrophy and myopathy. The results indicate Brg1 as a factor in the signaling pathways that generate cardiac hypertrophy and fibrosis, in part due to affecting the expression of myosin heavy chain. As a result it is possible to suppress or reduce cardiac hypertrophy by inhibiting Brg1 activity in cardiomyocytes. A Brg1 inhibitor may be useful as a cardiac hypertrophy suppressant and as a medicinal agent to prevent or remedy heart disease, when brought into contact with cardiac endothelial cells or cardiomyocytes. Conversely, it is possible to promote the onset of cardiac hypertrophy by increasing Brg1 activity in cardiomyocytes and/or endothelial cells, and create and provide animal disease models of cardiac hypertrophy.

The present invention provides a pharmaceutical composition effective in suppressing cardiac hypertrophy that has as an active ingredient a substance that suppresses the functional expression or activity in cardiomyocytes or cardiac endothelial cells of Brg1. The present invention provides a pharmaceutical composition that can suppress the onset and development of various types of heart disease caused by cardiac hypertrophy by using a substance that suppresses the related functional expression of BRG in order to block or suppress the cardiac hypertrophy signaling. The present invention further provides a method to suppress cardiac hypertrophy and to prevent onset of cardiac hypertrophy, as well as a method to prevent or remedy the onset and development of various kinds of heart disease such as chronic cardiac failure that are caused by the aforementioned cardiac hypertrophy.

In addition, the present invention provides a method, based on the newly discovered mechanisms of generating cardiac hypertrophy, that screens and selects hypertrophy suppressants and the components effective to remedy or prevent cardiac diseases caused by cardiac hypertrophy; and provides pharmaceutical compositions having the related components as the active ingredients (pharmaceutical compositions to suppress cardiac hypertrophy, pharmaceutical compositions to prevent or remedy cardiac diseases caused by cardiac hypertrophy).

The identification of Brg1 as a factor in cardiac hypertrophy provides diagnostic and prognostic methods, which detect the occurrence of a disorder, e.g. cardiomyopathy; atrial enlargement; myocardial hypertrophy; etc., particularly where such a disorder is indicative of a propensity for heart failure; or assess an individual's susceptibility to such disease, by detecting altered expression of Brg1 in cardiac endothelial cells and/or cardiomyocytes. Early detection of genes or their products can be used to determine the occurrence of developing disease, thereby allowing for intervention with appropriate preventive or protective measures.

Various techniques and reagents find use in the diagnostic methods of the present invention. In one embodiment of the invention, blood samples, or samples derived from blood, e.g. plasma, serum, etc. are assayed for the presence of polypeptides encoded by pressure overload associated genes, e.g. cell surface and, of particular interest, secreted polypeptides. In other embodiments a cell sample of heart tissue is analyzed for the presence of such polypeptides or mRNA encoding such polypeptides. Such polypeptides or mRNA may be detected through specific binding members. The use of antibodies for this purpose is of particular interest. Various formats find use for such assays, including antibody arrays; ELISA and RIA formats; binding of labeled antibodies in suspension/solution and detection by flow cytometry, mass spectroscopy, and the like. Detection may utilize one or a panel of antibodies.

Functional modulation of Brg1 in cardiac endothelial cells and/or cardiomyocytes provides a point of intervention to block the pathophysiologic processes of hypertrophy and enlargement, and also provides therapeutic intervention in other cardiovascular system diseases with similar pathophysiologies, to prevent, attenuate or reduce damage in prophylactic strategies in patients at high-risk of heart failure. The agent that acts to decrease such gene product activity can be an anti-sense or RNAi nucleic acid, neutralizing antibodies or any agent that acts as a direct or indirect inhibitor of the gene product, e.g. a pharmacological agonist, or partial agonist.

### DEFINITIONS

Heart failure is a general term that describes the final common pathway of many disease processes. Heart failure is usually caused by a reduction in the efficiency of cardiac muscle contraction. However, mechanical overload with normal or elevated cardiac contraction can also cause heart failure. This mechanical overload may be due to arterial hypertension, or stenosis or leakage of the aortic, mitral, or pulmonary valves, ischemic heart disease, congenital malformation of aorta, or pulmonary arteries, atrial or ventricular septal defects or other causes. The initial response to overload is usually hypertrophy (cellular enlargement) of the myocardium to increase force production, returning cardiac output (CO) to normal levels. Typically, a hypertrophic heart has impaired relaxation, a syndrome referred to as diastolic dysfunction. In the natural history of the disease, compensatory hypertrophy in the face of ongoing overload is followed by thinning, dilation, and enlargement, resulting in systolic dysfunction, also commonly known as heart failure. This natural progression typically occurs over the course of months to many years in humans, depending on the severity of the overload stimulus. Intervention at the hypertrophy stage can slow or prevent the progression to the clinically significant systolic dysfunction stage. Thus, diagnosis in the early hypertrophy stage provides unique therapeutic opportunities. The most common cause of congestive heart failure is coronary artery disease, which can cause a myocardial infarction (heart attack), which forces the heart to carry out the same work with fewer heart cells. The result is a pathophysiological state where the heart is unable to pump out enough blood to meet the nutrient and oxygen requirements of metabolizing tissues or cells. The phrase “manifestations of heart failure” is used broadly to encompass all of the sequelae associated with heart failure, such as shortness of breath, pitting edema, an enlarged tender liver, engorged neck veins, pulmonary rales and the like, including laboratory findings associated with heart failure.

In LV failure, CO declines and pulmonary venous pressure increases. Elevated pulmonary capillary pressure to levels that exceed the oncotic pressure of the plasma proteins (about 24 mm Hg) leads to increased lung water, reduced pulmonary compliance, and a rise in the O₂ cost of the work of breathing. Pulmonary venous hypertension and edema resulting from LV failure significantly alter pulmonary mechanics and, thereby, ventilation/perfusion relationships. When pulmonary venous hydrostatic pressure exceeds plasma protein oncotic pressure, fluid extravasates into the capillaries, the interstitial space, and the alveoli.

Increased heart rate and myocardial contractility, arteriolar constriction in selected vascular beds, venoconstriction, and Na and water retention compensate in the early stages for reduced ventricular performance. Adverse effects of these compensatory efforts include increased cardiac work, reduced coronary perfusion, increased cardiac preload and afterload, fluid retention resulting in congestion, myocyte loss, increased K excretion, and cardiac arrhythmia.

The mechanism by which an asymptomatic patient with cardiac dysfunction develops overt CHF is unknown, but it begins with renal retention of Na and water, secondary to decreased renal perfusion. Thus, as cardiac function deteriorates, renal blood flow decreases in proportion to the reduced CO, the GFR falls, and blood flow within the kidney is redistributed. The filtration fraction and filtered Na decrease, but tubular resorption increases.

Although symptoms and signs, for example exertional dyspnea, orthopnea, edema, tachycardia, pulmonary rales, a third heart sound, jugular venous distention, etc. have a diagnostic specificity of 70 to 90%, the sensitivity and predictive accuracy of conventional tests are low. Elevated levels of B-type natriuretic peptide may be diagnostic. Adjunctive tests include CBC, blood creatinine, BUN, electrolytes (eg, Mg, Ca), glucose, albumin, and liver function tests. ECG may be performed in all patients with HF, although findings are not specific.

As used herein, the term “cardiac hypertrophy” refers to the process in which adult cardiac myocytes respond to stress through hypertrophic growth. Such growth is characterized by cell size increasing without cell division, assembling of additional sarcomeres within the cell to maximize force generation, and an activation of a fetal cardiac gene program. Cardiac hypertrophy is often associated with increased risk of morbidity and mortality, and thus studies aimed at understanding the molecular mechanisms of cardiac hypertrophy have a significant impact on human health.

The term “treatment” or grammatical equivalents encompasses the improvement and/or reversal of the symptoms of heart failure (i.e., the ability of the heart to pump blood). “Improvement in the physiologic function” of the heart may be assessed using any of the measurements described herein (e.g., measurement of ejection fraction, fractional shortening, left ventricular internal dimension, heart rate, etc.), as well as any effect upon the animal's survival. In use of animal models, the response of treated transgenic animals and untreated transgenic animals is compared using any of the assays described herein (in addition, treated and untreated non-transgenic animals may be included as controls). A compound which causes an improvement in any parameter associated with heart failure used in the screening methods of the instant invention may thereby be identified as a therapeutic compound. Humans and other mammals may be targeted for the methods of the invention. The mammals in question are not particularly limited, and, concretely, may include rats, mice, hamsters, guinea pigs, dogs, monkeys, cows, horses, sheep, goats, and pigs, etc.

The term “compound” refers to any chemical entity, pharmaceutical, drug, and the like that can be used to treat or prevent a disease, illness, sickness, or disorder of bodily function. Compounds comprise both known and potential therapeutic compounds. A compound can be determined to be therapeutic by screening using the screening methods of the present invention. A “known therapeutic compound” refers to a therapeutic compound that has been shown (e.g., through animal trials or prior experience with administration to humans) to be effective in such treatment.

As used herein, the term “agonist” refers to molecules or compounds that mimic or enhance the action of a “native” or “natural” molecule. Agonists may include proteins, nucleic acids, carbohydrates, or any other molecules that interact with a molecule, receptor, and/or pathway of interest.

As used herein, the terms “antagonist” and “inhibitor” refer to molecules or compounds that inhibit the action of a cellular factor involved in cardiac hypertrophy. Antagonists and inhibitors may include proteins, nucleic acids, carbohydrates, or any other molecules which bind or interact with a receptor, molecule, and/or pathway of interest.

As used herein, the term “modulate” refers to a change or an alteration in the biological activity. Modulation may be an increase or a decrease in protein activity, a change in binding characteristics, or any other change in the biological, functional, or immunological properties associated with the activity of a protein or other structure of interest. The term “modulator” refers to any molecule or compound which is capable of changing or altering biological activity as described above.

### Disease Conditions, Diagnosis and Treatment

Cardiac hypertrophy is caused by increased load based on exercise, and disease factors such as increased pressure load based on hypertension, increased volume load based on valvular disorders, and increased load based on diseases of unknown cause. The cardiac hypertrophy of the present invention means the latter, specifically, myocardial disease conditions, such as compensatory hypertrophy of the heart and hypertrophic myocardial disease, in which the volume of the heart has increased beyond the range of physiological hypertrophy, based on various stresses such as hemodynamic overload and liquid factors by the disease condition.

Differences in hypertrophy of various parts of the heart, such as left ventricular hypertrophy, right ventricular hypertrophy, bilateral ventricular hypertrophy, and atrial hypertrophy may arise depending on the part where cardiac load is applied, but these types of hypertrophy are not particularly distinguished in the present invention. Moreover, if the overload applied to the heart is pressure load, there is a tendency for the wall thickness to increase notably and for the inner chamber to become deformed or narrowed (concentric hypertrophy); and if the overload applied to the heart is volume load, there is a tendency for the inner chamber to expand without that much increase in wall thickness (eccentric hypertrophy).

Hypertrophic cardiomyopathies are congenital or acquired disorders characterized by marked ventricular hypertrophy with diastolic dysfunction that may develop in the absence of increased afterload. The cardiac muscle is abnormal with cellular and myofibrillar disarray, although this finding is not specific to hypertrophic cardiomyopathy. The interventricular septum may be hypertrophied more than the left ventricular posterior wall (asymmetric septal hypertrophy). In the most common asymmetric form of hypertrophic cardiomyopathy, there is marked hypertrophy and thickening of the upper interventricular septum below the aortic valve. During systole, the septum thickens and the anterior leaflet of the mitral valve, already abnormally oriented due to the abnormal shape of the ventricle, is sucked toward the septum, producing outflow tract obstruction. Clinical manifestations may occur alone or in any combination: Chest pain is usually typical angina related to exertion. Syncope is usually exertional and due to a combination of cardiomyopathy, arrhythmia, outflow tract obstruction, and poor diastolic filling of the ventricle. Dyspnea on exertion results from poor diastolic compliance of the left ventricle, which leads to a rapid rise in left ventricular end-diastolic pressure as flow increases. Outflow tract obstruction, by lowering cardiac output, may contribute to the dyspnea.

The cardiac hypertrophy targeted by the present invention is induced via alterations in gene expression in cardiac endothelial cells and/or cardiomyocytes, particularly alterations associated with altered programs of myosin heavy chain gene expression, which relate to control by the BAF complex, particularly related to increased expression of Brg1.

### Therapeutic/Prophylactic Treatment Methods

Agents that modulate activity or expression of proteins in the BAF complex, particularly Brg1, provide a point of therapeutic or prophylactic intervention in the treatment of cardiac hypertrophy. Numerous agents are useful in modulating this activity, including agents that directly modulate expression, e.g. RNAi, antisense specific for the targeted gene; and agents that act on the protein, e.g. specific antibodies and analogs thereof, small organic molecules that block ATPase activity, etc. The genetic sequence of human Brg1 may be accessed at Genbank, locus NG_{—}011556, which listing is specifically incorporated by reference.

Antisense molecules can be used to down-regulate expression in cells. The antisense reagent may be antisense oligonucleotides (ODN), particularly synthetic ODN having chemical modifications from native nucleic acids, or nucleic acid constructs that express such antisense molecules as RNA. The antisense sequence is complementary to the mRNA of the targeted gene, and inhibits expression of the targeted gene products. Antisense molecules inhibit gene expression through various mechanisms, e.g. by reducing the amount of mRNA available for translation, through activation of RNAse H, or steric hindrance. One or a combination of antisense molecules may be administered, where a combination may comprise multiple different sequences.

Antisense molecules may be produced by expression of all or a part of the target gene sequence in an appropriate vector, where the transcriptional initiation is oriented such that an antisense strand is produced as an RNA molecule. Alternatively, the antisense molecule is a synthetic oligonucleotide. Antisense oligonucleotides will generally be at least about 7, usually at least about 12, more usually at least about 20 nucleotides in length, and not more than about 500, usually not more than about 50, more usually not more than about 35 nucleotides in length, where the length is governed by efficiency of inhibition, specificity, including absence of cross-reactivity, and the like.

Antisense oligonucleotides may be chemically synthesized by methods known in the art (see Wagner et al. (1993) supra. and Milligan et al., supra.) Preferred oligonucleotides are chemically modified from the native phosphodiester structure, in order to increase their intracellular stability and binding affinity. A number of such modifications have been described in the literature, which alter the chemistry of the backbone, sugars or heterocyclic bases.

In one embodiment of the invention, RNAi technology is used. As used herein, RNAi technology refers to a process in which double-stranded RNA is introduced into cells expressing a candidate gene to inhibit expression of the candidate gene, i.e., to “silence” its expression. The dsRNA is selected to have substantial identity with the candidate gene. In general such methods initially involve transcribing a nucleic acids containing all or part of a candidate gene into single- or double-stranded RNA. Sense and anti-sense RNA strands are allowed to anneal under appropriate conditions to form dsRNA. The resulting dsRNA is introduced into cells via various methods. Usually the dsRNA consists of two separate complementary RNA strands. However, in some instances, the dsRNA may be formed by a single strand of RNA that is self-complementary, such that the strand loops back upon itself to form a hairpin loop. Regardless of form, RNA duplex formation can occur inside or outside of a cell.

dsRNA can be prepared according to any of a number of methods that are known in the art, including in vitro and in vivo methods, as well as by synthetic chemistry approaches. Examples of such methods include, but are not limited to, the methods described by Sadher et al. (Biochem. Int. 14:1015, 1987); by Bhattacharyya (Nature 343:484, 1990); and by Livache, et al. (U.S. Pat. No. 5,795,715), each of which is incorporated herein by reference in its entirety. Single-stranded RNA can also be produced using a combination of enzymatic and organic synthesis or by total organic synthesis. The use of synthetic chemical methods enables one to introduce desired modified nucleotides or nucleotide analogs into the dsRNA. dsRNA can also be prepared in vivo according to a number of established methods (see, e.g., Sambrook, et al. (1989) Molecular Cloning: A Laboratory Manual, 2nd ed.; Transcription and Translation (B. D. Hames, and S. J. Higgins, Eds., 1984); DNA Cloning, volumes I and II (D. N. Glover, Ed., 1985); and Oligonucleotide Synthesis (M. J. Gait, Ed., 1984, each of which is incorporated herein by reference in its entirety).

A number of options can be utilized to deliver the dsRNA into a cell or population of cells, e.g. cardiac endothelial cells and/or cardiomyocytes. For instance, RNA can be directly introduced intracellularly. Various physical methods are generally utilized in such instances, such as administration by microinjection (see, e.g., Zernicka-Goetz, et al. (1997) Development 124:1133-1137; and Wianny, et al. (1998) Chromosoma 107: 430-439). Other options for cellular delivery include permeabilizing the cell membrane and electroporation in the presence of the dsRNA, liposome-mediated transfection, or transfection using chemicals such as calcium phosphate. A number of established gene therapy techniques can also be utilized to introduce the dsRNA into a cell. By introducing a viral construct within a viral particle, for instance, one can achieve efficient introduction of an expression construct into the cell and transcription of the RNA encoded by the construct.

Alternative agents include small molecules, peptides, antibodies and antibody fragments, etc., that interfere with the biological activity of Brg1. Identification of such molecules may be performed as described herein.

The pharmaceutical compositions for suppressing cardiac hypertrophy of the present invention comprise as active ingredients substances that suppress the functional activity or expression of Brg1 in cardiomyocytes and/or endothelial cells. Here, suppression includes both 100% suppression (inhibition) of the functional expression, or reduction of the activity of Brg1 without 100% inhibition. The substances may be ones that result in suppression of the functional expression of Brg1 in cardiomyocytes or endothelial cells, and the following may be cited as examples: substances that suppress the expression or production of Brg1 in cardiomyocytes/endothelial cells, substances that block or suppress Brg1 involvement in the BAF complex, and substances that suppress the ATPase activity of Brg1 in cardiomyocytes/endothelial cells, and the like.

These substances may be used in the form of pharmaceutical compositions at a dose effective to prevent or remedy heart diseases caused by cardiac hypertrophy together with pharmaceutically acceptable carriers and other additives. The pharmaceutical composition of the present invention may further contain well-known therapeutic drugs for heart disease as necessary. The therapeutic drugs for heart disease are not particularly limited, but β-blockers, anti-hypertensive agents, cardiotonic agents, anti-thrombosis agents, vasodilators, endothelial receptor blockers, calcium channel blockers, phosphodiesterase inhibitors, Angll receptor blockers, cytokine receptor blockers, gp130 receptor inhibitors, and the like.

The method to prevent or remedy heart disease caused by cardiac hypertrophy of the present invention may be carried out by administering to test subjects with heart diseases caused by cardiac hypertrophy or the preconditions thereof the effective amount of a substance that suppresses the functional activity or expression in cardiomyocytes or endothelial cells of Brg1. The method in question may be effectively used as a method to prevent cardiac hypertrophy from developing into heart disease for a test subject with cardiac hypertrophy.

### Diagnostic and Prognostic Methods

The differential expression of Brg1 in hypertrophic cardiac endothelial cells and/or cardiomyocytes provides for its use as a marker for diagnosis, and in prognostic evaluations to detect individuals at risk for cardiac pathologies, including atrial enlargement, ventricular hypertrophy, heart failure, etc. Prognostic methods can also be utilized to monitor an individual's health status prior to and after an episode, as well as in the assessment of the severity of the episode and the likelihood and extent of recovery.

In general, such diagnostic and prognostic methods involve detecting an altered level of expression of Brg1 mRNA or protein in the cells or tissue of an individual or a sample therefrom, to generate an expression profile. A variety of different assays can be utilized to detect an increase in Brg1 gene expression, including both methods that detect gene transcript and protein levels. More specifically, the diagnostic and prognostic methods disclosed herein involve obtaining a sample from an individual and determining at least qualitatively, and preferably quantitatively, the level of a Brg1 mRNA or protein expression in the sample. Usually this determined value or test value is compared against some type of reference or baseline value.

Profiles may be generated by any convenient means for determining differential gene expression between two samples, e.g. quantitative hybridization of mRNA, labeled mRNA, amplified mRNA, cRNA, etc., quantitative PCR, ELISA for protein quantitation, and the like.

The expression profile may be generated from a biological sample of cardiac endothelial cells and/or cardiomyocytes using any convenient protocol. A variety of different manners of generating expression profiles are known, such as those employed in the field of differential gene expression analysis. Following obtainment of the expression profile from the sample being assayed, the expression profile is compared with a reference or control profile to make a diagnosis regarding the susceptibility phenotype of the cell or tissue from which the sample was obtained/derived. Typically a comparison is made with a set of cells from an unaffected, normal source. Additionally, a reference or control profile may be a profile that is obtained from a cell/tissue known to be predisposed to heart failure, and therefore may be a positive reference or control profile.

In certain embodiments, the obtained expression profile is compared to a single reference/control profile to obtain information regarding the phenotype of the cell/tissue being assayed. In yet other embodiments, the obtained expression profile is compared to two or more different reference/control profiles to obtain more in depth information regarding the phenotype of the assayed cell/tissue. For example, the obtained expression profile may be compared to a positive and negative reference profile to obtain confirmed information regarding whether the cell/tissue has the phenotype of interest.

The difference values, i.e. the difference in expression in the presence and absence of radiation may be performed using any convenient methodology, where a variety of methodologies are known to those of skill in the array art, e.g., by comparing digital images of the expression profiles, by comparing databases of expression data, etc. Patents describing ways of comparing expression profiles include, but are not limited to, U.S. Pat. Nos. 6,308,170 and 6,228,575, the disclosures of which are herein incorporated by reference. Methods of comparing expression profiles are also described above. A statistical analysis step may then be performed to compare the expression profiles.

In one embodiment, an mRNA sample from heart tissue, preferably from one or more chambers affected by pressure overload, is analyzed for the genetic signature indicating overexpression of Brg1, and diagnostic of a tendency to heart failure. Expression signatures typically utilize a panel of genetic sequences, e.g. multiplex amplification, etc., coupled with analysis of the results to determine if there is a statistically significant match with a disease signature.

Nucleic acids or binding members such as antibodies that are specific for Brg1 polypeptides can be used to screen patient samples for increased expression of the corresponding mRNA or protein. Samples can be obtained from a variety of sources. For example, since the methods are designed primarily to diagnosis and assess risk factors for humans, samples are typically obtained from a human subject. However, the methods can also be utilized with samples obtained from various other mammals, such as primates, e.g. apes and chimpanzees, mice, cats, rats, and other animals. Such samples are referred to as a patient sample.

Samples can be obtained from the tissues or fluids of an individual, as well as from cell cultures or tissue homogenates. For example, samples can be obtained from whole blood, heart tissue biopsy of cardiac endothelial cells and/or cardiomyocytes, serum, etc. Also included in the term are derivatives and fractions of such cells and fluids. Where cells are analyzed, the number of cells in a sample will often be at least about 10², usually at least 10³, and may be about 10⁴ or more. The cells may be dissociated, in the case of solid tissues, or tissue sections may be analyzed. Alternatively a lysate of the cells may be prepared.

Diagnostic samples are collected any time after an individual is suspected to have cardiomyopathy, atrial enlargement, ventricular hypertrophy, etc. or has exhibited symptoms that predict such pathologies. In prophylactic testing, samples can be obtained from an individual who present with risk factors that indicate a susceptibility to heart failure, which risk factors include high blood pressure, obesity, diabetes, etc. as part of a routine assessment of the individual's health status.

The various test values determined for a sample from an individual believed to suffer pressure overload, cardiac hypertrophy, diastolic dysfunction, and/or a tendency to heart failure typically are compared against a baseline value to assess the extent of increased or decreased expression, if any. This baseline value can be any of a number of different values. In some instances, the baseline value is a value established in a trial using a healthy cell or tissue sample that is run in parallel with the test sample. Alternatively, the baseline value can be a statistical value (e.g., a mean or average) established from a population of control cells or individuals. For example, the baseline value can be a value or range that is characteristic of a control individual or control population. For instance, the baseline value can be a statistical value or range that is reflective of expression levels for the general population, or more specifically, healthy individuals not susceptible to stroke. Individuals not susceptible to stroke generally refer to those having no apparent risk factors correlated with heart failure, such as high blood pressure, high cholesterol levels, diabetes, smoking and high salt diet, for example.

### Nucleic Acid Screening Methods

Some of the diagnostic and prognostic methods that involve the detection of a Brg1 gene transcript begin with the lysis of cells and subsequent purification of nucleic acids from other cellular material, particularly mRNA transcripts. A nucleic acid derived from an mRNA transcript refers to a nucleic acid for whose synthesis the mRNA transcript, or a subsequence thereof, has ultimately served as a template. Thus, a cDNA reverse transcribed from an mRNA, an RNA transcribed from that cDNA, a DNA amplified from the cDNA, an RNA transcribed from the amplified DNA, are all derived from the mRNA transcript and detection of such derived products is indicative of the presence and/or abundance of the original transcript in a sample. Thus, suitable samples include, but are not limited to, mRNA transcripts of pressure overload associated genes, cDNA reverse transcribed from the mRNA, cRNA transcribed from the cDNA, DNA amplified from pressure overload associated nucleic acids, and RNA transcribed from amplified DNA.

A number of methods are available for analyzing nucleic acids for the presence of a specific sequence, e.g. upregulated expression. The nucleic acid may be amplified by conventional techniques, such as the polymerase chain reaction (PCR), to provide sufficient amounts for analysis. The use of the polymerase chain reaction is described in Saiki et al. (1985) Science 239:487, and a review of techniques may be found in Sambrook, et al. Molecular Cloning: A Laboratory Manual, CSH Press 1989, pp.14.2-14.33.

A detectable label may be included in an amplification reaction. Suitable labels include fluorochromes, e.g. fluorescein isothiocyanate (FITC), rhodamine, Texas Red, phycoerythrin, allophycocyanin,6-carboxyfluorescein(6-FAM),2,7-dimethoxy-4,5-dichloro-6-carboxyfluorescein (JOE), 6-carboxy-X-rhodamine (ROX), 6-carboxy-2,4,7,4,7-hexachlorofluorescein (HEX), 5-carboxyfluorescein (5-FAM) or N,N,N,N-tetramethyl-6-carboxyrhodamine (TAMRA), radioactive labels, e.g. ³²P, ³⁵S, ³H; etc. The label may be a two stage system, where the amplified DNA is conjugated to biotin, haptens, etc. having a high affinity binding partner, e.g. avidin, specific antibodies, etc., where the binding partner is conjugated to a detectable label. The label may be conjugated to one or both of the primers. Alternatively, the pool of nucleotides used in the amplification is labeled, so as to incorporate the label into the amplification product.

The sample nucleic acid, e.g. amplified, labeled, cloned fragment, etc. is analyzed by one of a number of methods known in the art. Probes may be hybridized to northern or dot blots, or liquid hybridization reactions performed. The nucleic acid may be sequenced by dideoxy or other methods, and the sequence of bases compared to a wild-type sequence. Single strand conformational polymorphism (SSCP) analysis, denaturing gradient gel electrophoresis (DGGE), and heteroduplex analysis in gel matrices are used to detect conformational changes created by DNA sequence variation as alterations in electrophoretic mobility. Fractionation is performed by gel or capillary electrophoresis, particularly acrylamide or agarose gels.

In situ hybridization methods are hybridization methods in which the cells are not lysed prior to hybridization. Because the method is performed in situ, it has the advantage that it is not necessary to prepare RNA from the cells. The method usually involves initially fixing test cells to a support (e.g., the walls of a microtiter well) and then permeabilizing the cells with an appropriate permeabilizing solution. A solution containing labeled probes for a pressure overload associated gene is then contacted with the cells and the probes allowed to hybridize with the nucleic acids. Excess probe is digested, washed away and the amount of hybridized probe measured. This approach is described in greater detail by Harris, D. W. (1996) Anal. Biochem. 243:249-256; Singer, et al. (1986) Biotechniques 4:230-250; Haase et al. (1984) Methods in Virology, vol. VII, pp. 189-226; and Nucleic Acid Hybridization: A Practical Approach (Hames, et al., eds., 1987).

A variety of so-called “real time amplification” methods or “real time quantitative PCR” methods can also be utilized to determine the quantity of pressure overload associated gene mRNA present in a sample. Such methods involve measuring the amount of amplification product formed during an amplification process. Fluorogenic nuclease assays are one specific example of a real time quantitation method that can be used to detect and quantitate pressure overload associated gene transcripts. In general such assays continuously measure PCR product accumulation using a dual-labeled fluorogenic oligonucleotide probe—an approach frequently referred to in the literature simply as the “TaqMan” method.

The probe used in such assays is typically a short (ca. 20-25 bases) polynucleotide that is labeled with two different fluorescent dyes. The 5′ terminus of the probe is typically attached to a reporter dye and the 3′ terminus is attached to a quenching dye, although the dyes can be attached at other locations on the probe as well. For measuring a pressure overload associated gene transcript, the probe is designed to have at least substantial sequence complementarity with a probe binding site on a pressure overload associated gene transcript. Upstream and downstream PCR primers that bind to regions that flank the pressure overload associated gene are also added to the reaction mixture.

When the probe is intact, energy transfer between the two fluorophors occurs and the quencher quenches emission from the reporter. During the extension phase of PCR, the probe is cleaved by the 5′ nuclease activity of a nucleic acid polymerase such as Taq polymerase, thereby releasing the reporter dye from the polynucleotide-quencher complex and resulting in an increase of reporter emission intensity that can be measured by an appropriate detection system.

### Polypeptide Screening Methods

Screening for expression of the subject sequences may be based on the functional or antigenic characteristics of the protein. Various immunoassays designed to quantitate Brg1 may be used in screening. Detection may utilize staining of cells or histological sections, performed in accordance with conventional methods, using antibodies or other specific binding members that specifically bind to the pressure overload associated polypeptides. The antibodies or other specific binding members of interest, e.g. receptor ligands, are added to a cell sample, and incubated for a period of time sufficient to allow binding to the epitope, usually at least about 10 minutes. The antibody may be labeled with radioisotopes, enzymes, fluorescers, chemiluminescers, or other labels for direct detection. Alternatively, a second stage antibody or reagent is used to amplify the signal. Such reagents are well known in the art. For example, the primary antibody may be conjugated to biotin, with horseradish peroxidase-conjugated avidin added as a second stage reagent. Final detection uses a substrate that undergoes a color change in the presence of the peroxidase. The absence or presence of antibody binding may be determined by various methods, including flow cytometry of dissociated cells, microscopy, radiography, scintillation counting, etc.

An alternative method for diagnosis depends on the in vitro detection of binding between antibodies and Brg1 in a blood sample, cell lysate, etc. Measuring the concentration of the target protein in a sample or fraction thereof may be accomplished by a variety of specific assays. A conventional sandwich type assay may be used. For example, a sandwich assay may first attach specific antibodies to an insoluble surface or support. The particular manner of binding is not crucial so long as it is compatible with the reagents and overall methods of the invention. They may be bound to the plates covalently or non-covalently, preferably non-covalently.

After the second binding step, the insoluble support is again washed free of non-specifically bound material, leaving the specific complex formed between the target protein and the specific binding member. The signal produced by the bound conjugate is detected by conventional means. Where an enzyme conjugate is used, an appropriate enzyme substrate is provided so a detectable product is formed.

Other immunoassays are known in the art and may find use as diagnostics. Ouchterlony plates provide a simple determination of antibody binding. Western blots may be performed on protein gels or protein spots on filters, using a detection system specific for the pressure overload associated polypeptide as desired, conveniently using a labeling method as described for the sandwich assay.

The detection methods can be provided as part of a kit. Thus, the invention further provides kits for detecting the presence of a Brg1 mRNA or a polypeptide encoded thereby, in a biological sample. Procedures using these kits can be performed by clinical laboratories, experimental laboratories, medical practitioners, or private individuals. The kits of the invention for detecting a polypeptide comprise a moiety that specifically binds the polypeptide, which may be a specific antibody. The kits of the invention for detecting a nucleic acid comprise a moiety that specifically hybridizes to such a nucleic acid. The kit may optionally provide additional components that are useful in the procedure, including, but not limited to, buffers, developing reagents, labels, reacting surfaces, means for detection, control samples, standards, instructions, and interpretive information.

### Time Course Analyses

Certain prognostic methods of assessing a patient's risk of heart failure involve monitoring expression levels for a patient susceptible to heart failure, to track whether there is a change in gene expression over time. An increase in expression over time can indicate that the individual is at increased risk for heart failure. As with other measures, the expression level for the patient at risk for heart failure is compared against a baseline value. The baseline in such analyses can be a prior value determined for the same individual or a statistical value (e.g., mean or average) determined for a control group (e.g., a population of individuals with no apparent neurological risk factors). An individual showing a statistically significant increase in pressure overload associated expression levels over time can prompt the individual's physician to take prophylactic measures to lessen the individual's potential for heart failure. For example, the physician can recommend certain life style changes (e.g., medication, improved diet, exercise program) to reduce the risk of heart failure.

Patients diagnosed as being at risk for heart failure by the methods of the invention may be appropriately treated to reduce the risk of heart failure. Drug treatment of systolic dysfunction primarily involves diuretics, ACE inhibitors, ARB (angiotensin receptor blocker), aldosterone antagonists, digitalis, and β-blockers; most patients are treated with at least two of these classes. Addition of hydralazine and isosorbide dinitrate to standard triple therapy of HF may improve hemodynamics and exercise tolerance and reduce mortality in refractory patients. The angiotensin II receptor blocker losartan has effects similar to those of ACE inhibitors. Spironolactone antagonizes aldosterone effects and improves heart failure symptoms and survival.

Digitalis preparations have many actions, including weak inotropism, and blockade of the atrioventricular node. Digoxin is the most commonly prescribed digitalis preparation. Digitoxin, an alternative in patients with known or suspected renal disease, is largely excreted in the bile and is thus not influenced by abnormal renal function.

With careful administration of β-blockers, some patients, especially those with idiopathic dilated cardiomyopathy, will improve clinically and may have reduced mortality. Carvedilol, a 3rd-generation nonselective β-blocker, is also a vasodilator with a blockade and an antioxidant activity. Vasodilators such as nitroglycerin or nitroprusside improve ventricular function by reducing systolic ventricular wall stress, aortic impedance, ventricular chamber size, and valvular regurgitation.

Alternatively a patient diagnosed with cardiac hypertrophy or may be treated with an inhibitor of Brg1 as previously described.

### Compound Screening

Compound screening may be performed using an in vitro model, a genetically altered cell or animal, or purified protein corresponding to BAF, Brg1, etc. One can identify ligands or substrates that bind to, inhibit, modulate or mimic the action of the encoded polypeptide.

The polypeptides include those encoded by the provided genetic sequences, as well as nucleic acids that, by virtue of the degeneracy of the genetic code, are not identical in sequence to the disclosed nucleic acids, and variants thereof. Variant polypeptides can include amino acid (aa) substitutions, additions or deletions. The amino acid substitutions can be conservative amino acid substitutions or substitutions to eliminate non-essential amino acids, such as to alter a glycosylation site, a phosphorylation site or an acetylation site, or to minimize misfolding by substitution or deletion of one or more cysteine residues that are not necessary for function. Variants can be designed so as to retain or have enhanced biological activity of a particular region of the protein (e.g., a functional domain and/or, where the polypeptide is a member of a protein family, a region associated with a consensus sequence). Variants also include fragments of the polypeptides disclosed herein, particularly biologically active fragments and/or fragments corresponding to functional domains. Fragments of interest will typically be at least about 10 aa to at least about 15 aa in length, usually at least about 50 aa in length, and can be as long as 300 aa in length or longer, but will usually not exceed about 500 aa in length, where the fragment will have a contiguous stretch of amino acids that is identical to a polypeptide encoded by a pressure overload associated gene, or a homolog thereof.

Transgenic animals or cells derived therefrom are also used in compound screening. Transgenic animals may be made through homologous recombination, where the normal locus corresponding to a BAF gene is altered, as set forth in the Examples. Alternatively, a nucleic acid construct is randomly integrated into the genome. For example, increased expression of Brg1 may be induced in cardiac endothelial cells and/or cardiomyocytes. Vectors for stable integration include plasmids, retroviruses and other animal viruses, YACs, and the like. A series of small deletions and/or substitutions may be made in the coding sequence to determine the role of different domains. Specific constructs of interest include antisense sequences that block expression of the targeted gene, knockout, and expression of dominant negative mutations. A detectable marker, such as lac Z may be introduced into the locus of interest, where up-regulation of expression will result in an easily detected change in phenotype. One may also provide for expression of the target gene or variants thereof in cells or tissues where it is not normally expressed or at abnormal times of development. By providing expression of the target protein in cells in which it is not normally produced, one can induce changes in cell behavior.

Transgenic animals of interest include conditional knockout animals, where the expression of a BAF complex gene, e.g. Brg1, is selectively deleted in cardiac endothelial cells and/or cardiomyocytes. For example, a selectively expressed Cre recombinase will delete floxed genes, such as Brg1, in targeted tissues. In some embodiments the Sm22α transgene is used to target a gene for deletion in myocardial tissues.

In addition to cell-free screening methods, compounds may be tested in cells and the effect on expression of myosin heavy chain isotypes determined. Alternatively the activity or expression of Brg1 may be directly measured. Alternative screening methods may determine the effectiveness of an agent on the development of cardiac hypertrophy in an animal model, as described in the Examples.

Compound screening identifies agents that modulate function of Brg1. Of particular interest are screening assays for agents that have a low toxicity for human cells. A wide variety of assays may be used for this purpose, including labeled in vitro protein-protein binding assays, electrophoretic mobility shift assays, immunoassays for protein binding, and the like. Knowledge of the 3-dimensional structure of the encoded protein, derived from crystallization of purified recombinant protein, could lead to the rational design of small drugs that specifically inhibit activity. These drugs may be directed at specific domains.

The term “agent” as used herein describes any molecule, e.g. protein or pharmaceutical, with the capability of altering or mimicking the physiological function of a pressure overload associated associated gene. Generally a plurality of assay mixtures are run in parallel with different agent concentrations to obtain a differential response to the various concentrations. Typically one of these concentrations serves as a negative control, i.e. at zero concentration or below the level of detection.

Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Test agents can be obtained from libraries, such as natural product libraries or combinatorial libraries, for example. A number of different types of combinatorial libraries and methods for preparing such libraries have been described, including for example, PCT publications WO 93/06121, WO 95/12608, WO 95/35503, WO 94/08051 and WO 95/30642, each of which is incorporated herein by reference.

Where the screening assay is a binding assay, one or more of the molecules may be joined to a label, where the label can directly or indirectly provide a detectable signal. Various labels include radioisotopes, fluorescers, chemiluminescers, enzymes, specific binding molecules, particles, e.g. magnetic particles, and the like. Specific binding molecules include pairs, such as biotin and streptavidin, digoxin and antidigoxin, etc. For the specific binding members, the complementary member would normally be labeled with a molecule that provides for detection, in accordance with known procedures.

A variety of other reagents may be included in the screening assay. These include reagents like salts, neutral proteins, e.g. albumin, detergents, etc that are used to facilitate optimal protein-protein binding and/or reduce non-specific or background interactions. Reagents that improve the efficiency of the assay, such as protease inhibitors, nuclease inhibitors, anti-microbial agents, etc. may be used. The mixture of components are added in any order that provides for the requisite binding. Incubations are performed at any suitable temperature, typically between 4 and 40° C. Incubation periods are selected for optimum activity, but may also be optimized to facilitate rapid high-throughput screening. Typically between 0.1 and 1 hours will be sufficient.

Preliminary screens can be conducted by screening for compounds capable of binding to a BAF protein, as at least some of the compounds so identified are likely inhibitors. The binding assays usually involve contacting a protein with one or more test compounds and allowing sufficient time for the protein and test compounds to form a binding complex. Any binding complexes formed can be detected using any of a number of established analytical techniques. Protein binding assays include, but are not limited to, methods that measure co-precipitation, co-migration on non-denaturing SDS-polyacrylamide gels, and co-migration on Western blots. The protein utilized in such assays can be naturally expressed, cloned or synthesized.

Compounds that are initially identified by any of the foregoing screening methods can be further tested to validate the apparent activity. The basic format of such methods involves administering a lead compound identified during an initial screen to an animal that serves as a model for humans and then determining if the gene is in fact differentially regulated. The animal models utilized in validation studies generally are mammals. Specific examples of suitable animals include, but are not limited to, primates, mice, and rats.

In vivo assays involve the use of various animal models of heart disease, including transgenic animals, that have been engineered to have specific defects, or carry markers that can be used to measure the ability of a candidate substance to reach and effect different cells within the organism. Due to their size, case of handling, and information on their physiology and genetic make-up, mice are a preferred embodiment, especially for transgenics. However, other animals are suitable as well, including rats, rabbits, hamsters, guinea pigs, gerbils, woodchucks, cats, dogs, sheep, goats, pigs, cows, horses and monkeys (including chimps, gibbons and baboons). Assays for inhibitors may be conducted using an animal model derived from any of these species.

Treatment of animals with test compounds will involve the administration of the compound, in an appropriate form, to the animal. Administration will be by any route that could be utilized for clinical purposes. Determining the effectiveness of a compound in vivo may involve a variety of different criteria, including but not limited to. Also, measuring toxicity and dose response can be performed in animals in a meaningful fashion.

Active test agents identified by the screening methods described herein can serve as lead compounds for the synthesis of analog compounds. Typically, the analog compounds are synthesized to have an electronic configuration and a molecular conformation similar to that of the lead compound. Identification of analog compounds can be performed through use of techniques such as self-consistent field (SCF) analysis, configuration interaction (CI) analysis, and normal mode dynamics analysis. Computer programs for implementing these techniques are available. See, e.g., Rein et al., (1989) Computer-Assisted Modeling of Receptor-Ligand Interactions (Alan Liss, N.Y.).

Once analogs have been prepared, they can be screened using the methods disclosed herein to identify those analogs that exhibit an increased ability to modulate gene product activity. Such compounds can then be subjected to further analysis to identify those compounds that appear to have the greatest potential as pharmaceutical agents. Alternatively, analogs shown to have activity through the screening methods can serve as lead compounds in the preparation of still further analogs, which can be screened by the methods described herein. The cycle of screening, synthesizing analogs and re-screening can be repeated multiple times.

Compounds identified by the screening methods described above and analogs thereof can serve as the active ingredient in pharmaceutical compositions formulated for the treatment of various disorders, including a propensity for heart failure. The compositions can also include various other agents to enhance delivery and efficacy. The compositions can also include various agents to enhance delivery and stability of the active ingredients.

Thus, for example, the compositions can also include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

The composition can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, enhance solubility or uptake). Examples of such modifications or complexing agents include sulfate, gluconate, citrate and phosphate. The polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

The pharmaceutical compositions can be administered for prophylactic and/or therapeutic treatments. Toxicity and therapeutic efficacy of the active ingredient can be determined according to standard pharmaceutical procedures in cell cultures and/or experimental animals, including, for example, determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred.

The data obtained from cell culture and/or animal studies can be used in formulating a range of dosages for humans. The dosage of the active ingredient typically lines within a range of circulating concentrations that include the ED₅₀ with little or no toxicity. The dosage can vary within this range depending upon the dosage form employed and the route of administration utilized.

The pharmaceutical compositions described herein can be administered in a variety of different ways. Examples include administering a composition containing a pharmaceutically acceptable carrier via oral, intranasal, rectal, topical, intraperitoneal, intravenous, intramuscular, subcutaneous, subdermal, transdermal, and intrathecal methods.

Formulations suitable for parenteral administration, such as, for example, by intraarticular (in the joints), intravenous, cardiovascular, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

### Culture of Cardiomyocyte Progenitors

Proliferation and differentiation are two important cellular events that are generally inversely regulated. The BAF complex provides for reveal independent but coordinated control mechanisms for each through distinct pathways of BMP10 and HDACs/PARPs in developing progenitor cells. The high-ranking regulator, BAF, initiates parallel paths to determine the cellular fates of developing myocardial cells. Specifically, cells in which Brg1 is upregulated skew myosin heavy expression to the β-chain, and continue in a proliferative mode. The cells are induced to differentiate and cease proliferation by decreased Brg1 expression. Furthermore, cardiomyocyte differentiation can be induced by inhibition of the HDAC or PARP activity. For various purposes it is desirable to manipulate proliferation and differentiation of cardiomyocyte and cardiomyocyte progenitors during in vitro culture, which is accomplished by providing the cells with agents that enhance or inhibit Brg1 expression and/or function. Examples of such agents, including genetic agents encoding Brg1, anti-sense or RNAi agents that are complementary to Brg 1 and inhibit its activity, are described herein. Similarly, HDAC or PARP activity can be manipulated by genetic methods, RNAi agents or chemicals such as PJ-34.

In one embodiment there is provided a method of modulating cardiomyocyte differentiation of a human stem or progenitor cell, the method comprising culturing the stem or progenitor cell in the presence of an agent that alters Brg1 activity or expression, where increased cell proliferation is found when Brg1 activity or expression is increased. In another aspect the invention also provides for improving yield of cardiomyocytes and cardiac progenitors by adopting the methods described herein. “Enhancing cardiomyocyte differentiation” can include increasing the number of cardiomyocytes differentiated in a culture compared with a culture that is not enhanced and improving the efficiency of the cardiomyocyte differentiation process. Hence the induction of differentiation is improved over baseline levels. “Enhancing” can also include inducing the cardiomyocyte from an undifferentiated stem cell culture that is capable of cardiomyocyte differentiation.

The present invention also provides transgenic cardiomyocytes and cardiac progenitors as well as enriched transgenic cardiomyocyte populations and cardiac progenitor populations prepared by the methods of the present invention. In another aspect the invention includes a method of repairing cardiac tissue, the method including transplanting an isolated cardiomyocyte or cardiac progenitor cell of the invention into damaged cardiac tissue of a subject.

In some embodiments the culturing conditions are serum-free conditions. The periods in which the conditions are serum free are ideally from the time of culture of the stem cells or as part of the co-culture of the stem cells. In other embodiments the medium comprises serum. Agents known to induce cardiomyocyte differentiation may be included in the medium, e.g. following proliferation, or in combination with a Brg1 inducing agent.

Optionally, cardiotropic factors are included, as described in U.S. Patent application 20030022367, are added to the culture. Such factors may include nucleotide analogs that affect DNA methylation and alter expression of cardiomyocyte-related genes; TGF-β ligands, such as activin A, activin B, insulin-like growth factors, bone morphogenic proteins, fibroblast growth factors, platelet-derived growth factor natriuretic factors, insulin, leukemia inhibitory factor (LIF), epidermal growth factor (EGF), TGFα, and products of the cripto gene; antibodies and peptidomimetics with agonist activity for the same receptors, cells secreting such factors, and the like.

The period over which cardiomyocyte differentiation is induced may be at least 6 days. The period may be 6 to 12 days. The concentration of the serum may therefore be changed over this period. For instance some of the period may be in the presence of serum, and the remaining period may be in the absence of serum.

The term “inducing cardiomyocyte differentiation” as used herein is taken to mean causing a human stem cell or progenitor cell to develop into a cell of the cardiac lineage as a result of a direct or intentional influence on the stem cell. Influencing factors that may induce differentiation in a stem cell can include cellular parameters such as ion influx, a pH change and/or extracellular factors, such as secreted proteins, such as but not limited to growth factors and cytokines that regulate and trigger differentiation. Cells of the cardiac lineage include, but are not limited to cardiomyocytes and cardiac progenitors.

In the present invention a human stem cell is undifferentiated prior to culturing and is capable of undergoing differentiation. The stem cell may be selected from the group including, but not limited to, embryonic stem cells, pluripotent stem cells including embryonic stem cells and induced pluripotent stem cells, haematopoietic stem cells, totipotent stem cells, mesenchymal stem cells, neural stem cells, or adult stem cells.

The stem cells suitable for use in the present methods may be derived from a patient's own tissue. This would enhance compatibility of differentiated tissue grafts derived from the stem cells with the patient. The stem cells may be genetically modified prior to use through introduction of genes that may control their state of differentiation prior to, during or after their exposure to methods of the invention. They may be genetically modified through introduction of vectors expressing a selectable marker under the control of a stem cell specific promoter such as Oct-4 or of genes that may be upregulated to induce cardiomyocyte differentiation. The stem cells may be genetically modified at any stage with markers or gene so that the markers or genes are carried through to any stage of culturing. The markers may be used to purify or enrich the differentiated or undifferentiated stem cell populations at any stage of culture.

The cardiomyocytes and cardiac progenitors of the invention may be beating. Cardiomyocytes and the cardiac progenitors can be fixed and stained with α-actinin antibodies to confirm muscle phenotype. α-troponin, α-tropomysin and MHC antibodies also give characteristic muscle staining.

The cell composition of the present invention can be used in methods of repairing or treating diseases or conditions, such as cardiac disease or where tissue damage has occurred. The treatment may include, but is not limited to, the administration of cells or cell compositions (either as partly or fully differentiated) into patients. These cells or cell compositions would result in reversal of the condition via the restoration of cardiomyocyte function.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### EXAMPLE 1

Cardiac hypertrophy and failure are characterized by transcriptional reprogramming and fetal gene activation. Stressed adult hearts undergo a shift of myosin heavy chain (MHC) from adult α-MHC to fetal β-MHC isoform in mice, resulting in decreased cardiac contractility. However, common mechanisms bridging these developmental and pathological processes are not well understood. Here we show that Brg1, a core component of BAF chromatin-remodeling complex, plays critical roles in regulating gene expression, tissue growth and differentiation in embryonic hearts and adult hearts under stress. In embryos, Brg1 promotes myocardial proliferation by maintaining BMP10 and suppressing a CDK inhibitor, p57kip2. In parallel, Brg1/BAF preserves fetal differentiation by interacting with HDACs and PARP1 to repress α-MHC and activate β-MHC. Though highly expressed in embryos, Brg1 is turned off in adult myocardium. It is reactivated by cardiac stresses to complex with HDACs and PARP1, thus inducing pathological α/β-MHC shift. Preventing Brg1 re-expression decreases hypertrophy, and reverses α/β-MHC expression. Brg1 level is elevated in human hypertrophic cardiomyopathy; correlating with disease severity and MHC changes. This provides for the use of BAF as a therapeutic target for hypertrophic heart disease.

Cardiac contractility depends on heart muscle mass and its myosin content. The amount of heart muscle is primarily determined by the proliferation of embryonic myocardial cells before these cells undergo terminal differentiation and lose their proliferative capacity during the neonatal period. The balance between proliferation and differentiation is a highly regulated process in embryonic development, involving extensive signaling and transcriptional programs. Actively proliferating embryonic myocardial cells and post-mitotic adult cardiomyocytes have different contractile properties due to expression of different isoforms of myosin heavy chain (MHC), a molecular motor that hydrolyzes ATP to do mechanical work. Two MHC isoforms, α- and β-MHC, exist in the mammalian heart. The α-MHC has higher ATPase activity than the βisoform, and their amount changes under different developmental and pathophysiological conditions. The relative distribution of α- and β-MHC isoforms has been shown to be directly related to overall cardiac performance in animals as well as in patients with cardiac hypertrophy and failure. Also, structural changes of MHC proteins are sufficient to cause cardiac dysfunction in mice and humans. Pathologic hypertrophy is associated with the induction of β-MHC at the expense of α-MHC; however, transgenic studies indicate that hearts expressing α-MHC have better outcome under stress conditions, than the controls expressing mainly β-MHC. Thus, strategies to control MHC isoform expression in hypertrophic hearts represent attractive approaches for heart failure therapy.

Deciphering the mechanisms that control MHC expression in developmental and pathophysiological conditions remains of great interests in research and can provide a basis for therapeutic intervention. Chromatin remodeling offers one such control to modulate gene expression through changing the composition of histones, altering histone-DNA interactions or physically interacting with transcription factors. These changes modify the local chromatin structure, thereby changing the access of transcription and other associated factors to specific genes. The Brg1/Brm-associated-factor (BAF) complex is a SWI/SNF ATP-dependent chromatin remodeling complex, in which two genes, Brg1 and Brm, encode the ATPase component. Although both genes are widely expressed, Brg1^{-/-} mice die before E7.5 while Brm knockouts are viable, indicating that Brg1 is a non-redundant component of BAF complexes and that removing this single subunit will effectively render the whole complex nonfunctional.

BAF complexes are composed of twelve different protein subunits that are expressed ubiquitously or tissue-specifically, conferring tissue or target specific functions to the complex. Consistent with the heterogeneity of the BAF complex, previous studies have revealed the roles of Brg1 in T lymphocyte differentiation, limb development, and hematopoiesis.

Our studies reveal a new role of the BAF complex as a common link between developmental and pathological programming of cardiac gene expression. The BAF complex balances embryonic myocardial growth and differentiation and activates similar embryonic mechanisms in adults under cardiac stress to trigger pathological cardiac remodeling. We show that the BAF complex controls myocardial proliferation by activating morphogenic protrein-10 (BMP10) to suppress p57kip2, a cyclin-dependent kinase (CDK) inhibitor. Through a separate molecular pathway, the BAF complex controls myocardial MHC differentiation by interacting with other chromatin remodellers, histone deacetylases (HDACs) and poly (ADP-ribose) polymerase 1 (PARP1). Likewise, we found that BAF activation by cardiac stress is required for hypertrophic growth and MHC changes of adult murine hearts, and Brg1 activation in human hypertrophic cardiomyopathy correlates with the severity of hypertrophy and MHC changes in patients. In conclusion, the BAF complex provides a molecular mechanism that bridges myocardial growth and differentiation in embryos with those in the adult myocardium. The activation of Brg1/BAF in adult hearts can contribute to the development of human cardiomyopathy.

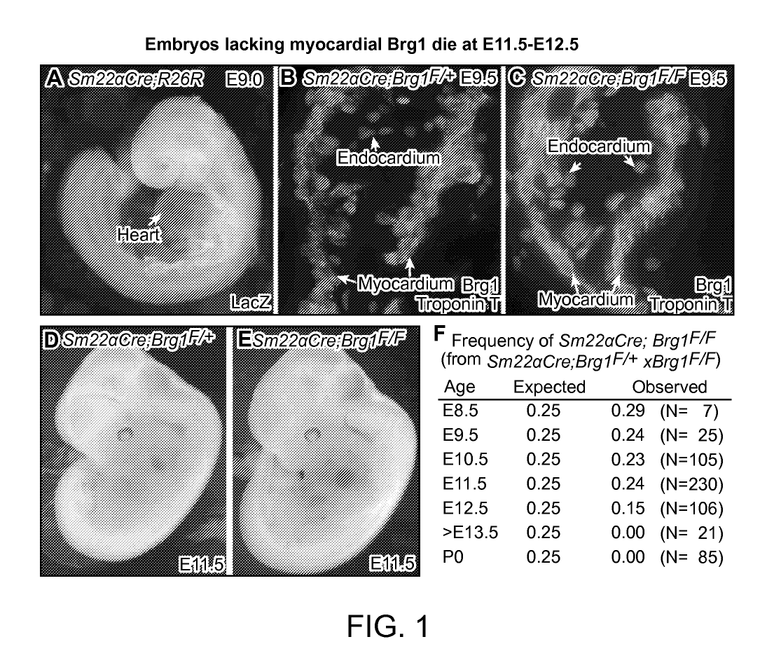

Myocardial deletion of Brg1 results in embryonic lethality after E11.5 To study the role of Brg1 in myocardial development, we used the Sm22α transgene to remove Brg1 in the myocardium of mice homozygous for a floxed allele of Brg1 (Brg1F). Sm22αCre expresses Cre recombinase transiently in embryonic myocardium as early as E9.0 (FIG. 1A). And by E9.5 Brg1 was specifically deleted in nearly all cells of Sm22αCre;Brg1^{F/F} myocardium, while retaining expression in the endocardium (FIGS. 1B and 1C). Sm22αCre;Brg1^{F/F} embryos were alive and morphologically comparable to littermate controls up to E11.5 (FIGS. 1D and 1E); they died after E11.5 with none surviving to E13.5 (FIG. 1F).

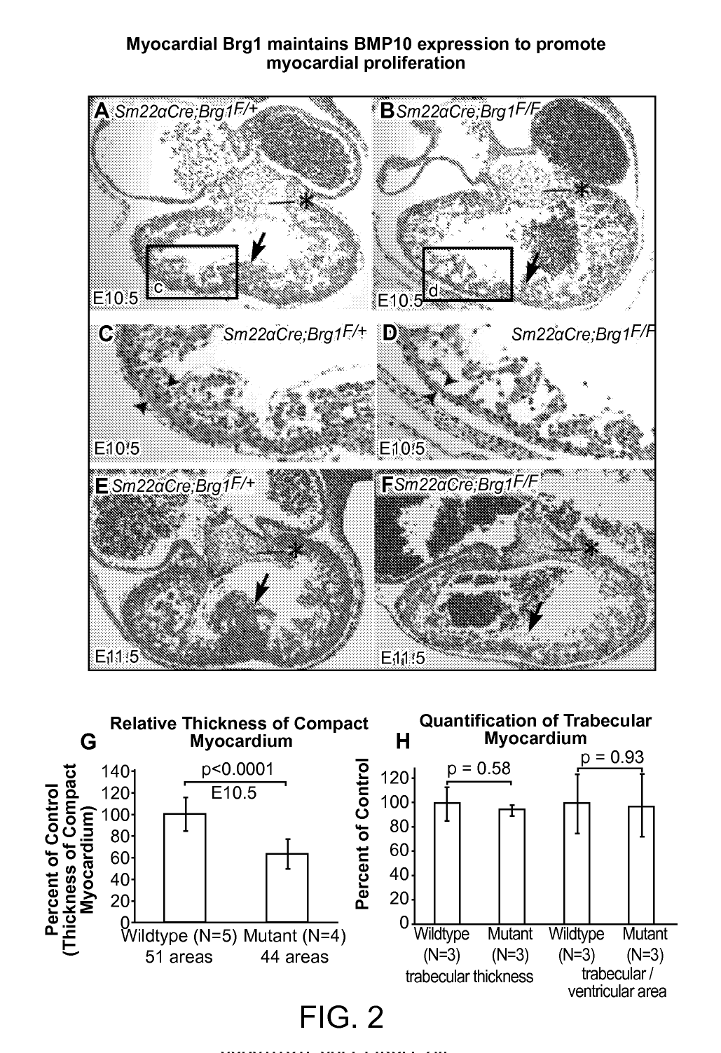

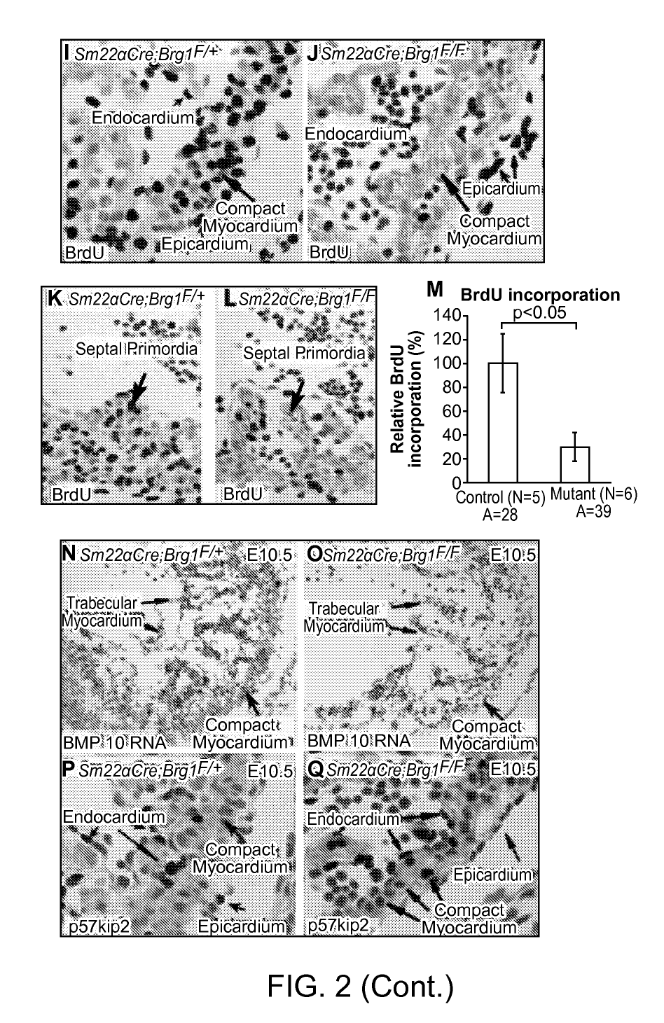

Brg1 is required in the myocardium for the formation of compact and septal myocardium To investigate the cause of embryonic lethality, we analyzed vasculature and heart development in Sm22αCre;Brg1^{F/F} embryos. There were no defects in the yolk sac or embryo vasculature by direct inspection and by whole mount PECAM staining (data not shown). In contrast, the compact myocardium in Sm22αCre;Brg1^{F/F} embryos was significantly thinner than in control littermates by E10.5 (FIGS. 2A-2D). Its thickness was approximately 60% of the controls (FIG. 2G). Furthermore, there was no muscular interventricular septum that separates the right and left ventricles (FIGS. 2B and 2F). However, the trabeculation of Sm22αCre;Brg1^{F/F}embryos appeared normal (FIGS. 2B and 2F), as measured by trabecular thickness and area normalized to the length of the compact wall (FIG. 2H). The endocardial cushion of Sm22αCre;Brg1^{F/F} embryos were also normal (FIGS. 2B and 2F). These findings suggest that BAF complexes have a tissue-specific role in myocardial development rather than broad effects on the whole heart between E9.0 and E11.5. The failure of myocardial thickening is sufficient to reduce the cardiac output, causing early lethality of Sm22αCre;Brg1^{F/F} embryos.

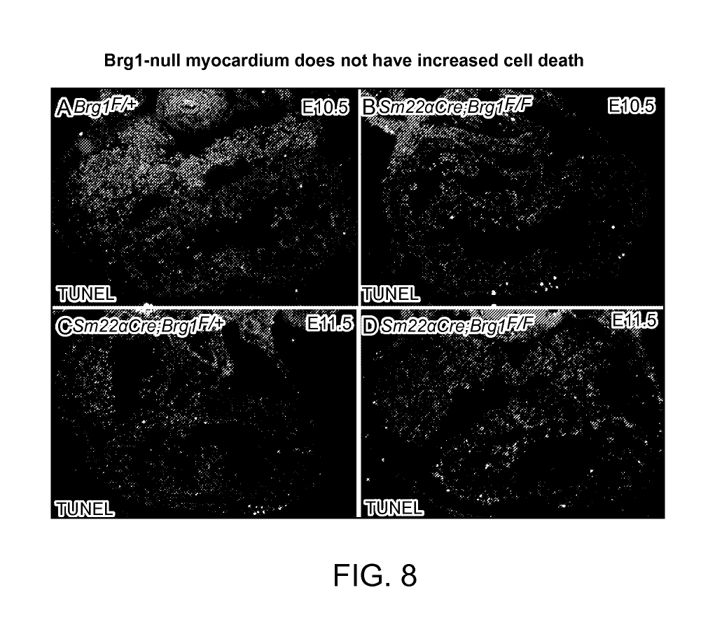

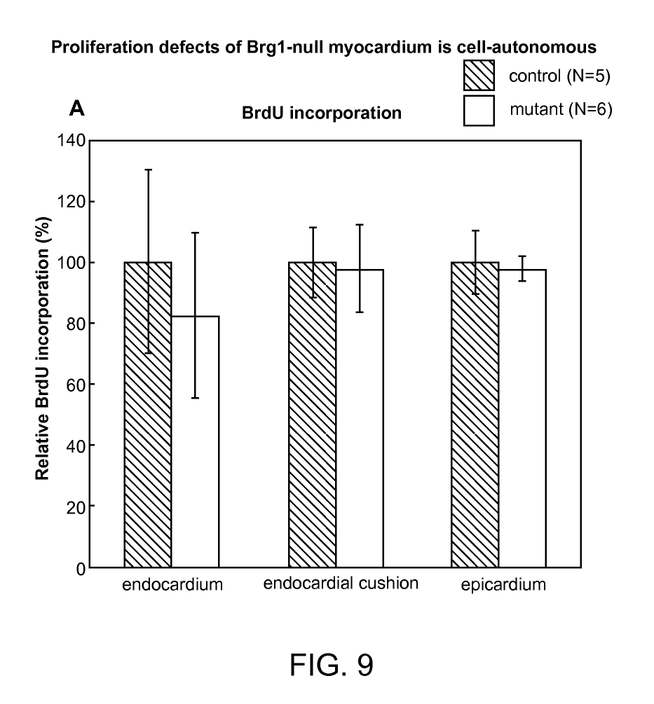

The compact myocardium and septal primordia fail to proliferate without Brg1 We next measured myocardial proliferation and cell death rates in Sm22αCre;Brg1^{F/F} embryos to ascertain if defects in these processes accounted for the failure of myocardial thickening. By TUNEL staining, we found almost no myocardial apoptosis in control and Sm22αCre;Brg1^{F/F} embryos (FIG. 8A-8D). In contrast, using bromodeoxyuridine (BrdU) to label cells that had gone through the S phase of cell cycle, we observed a dramatic decrease of cell proliferation by 70% (100+/−11% in control vs 32+/−16% in mutant embryos) in both the compact and the septal primordial myocardium of E10.5 Sm22αCre;Brg1^{F/F} embryos (FIGS. 2I-2M). However, BrdU incorporation in the endocardium, epicardium or endocardial cushion mesenchyme was normal in Sm22αCre;Brg1^{F/F} hearts (FIG. 9). These findings indicate that myocardial Brg1 is specifically required for cell proliferation in the myocardium, and disruption of this process causes the thin myocardium and failure of ventricular septal formation.

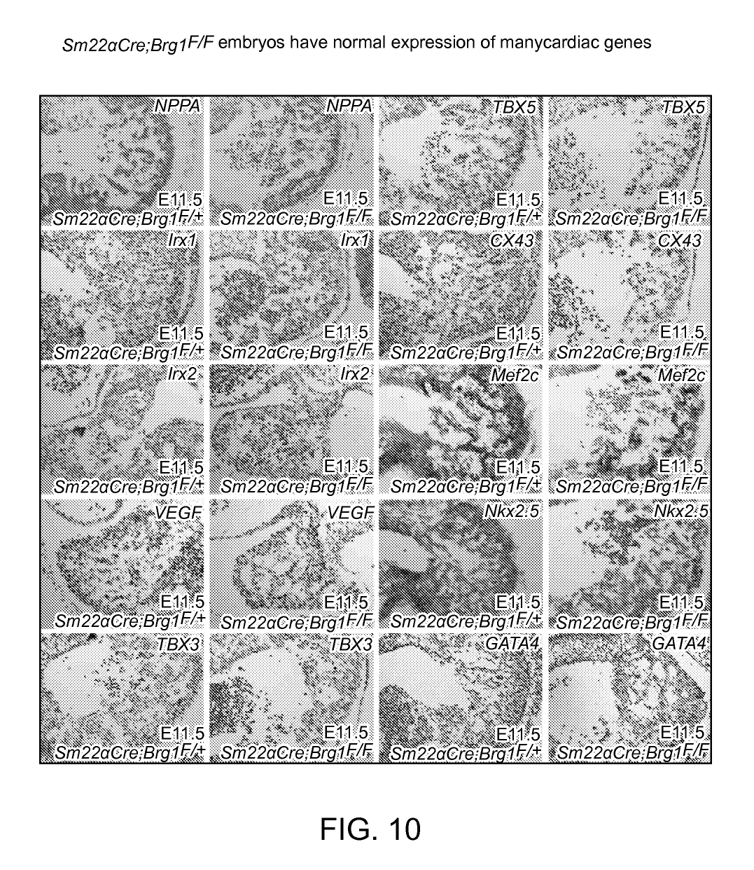

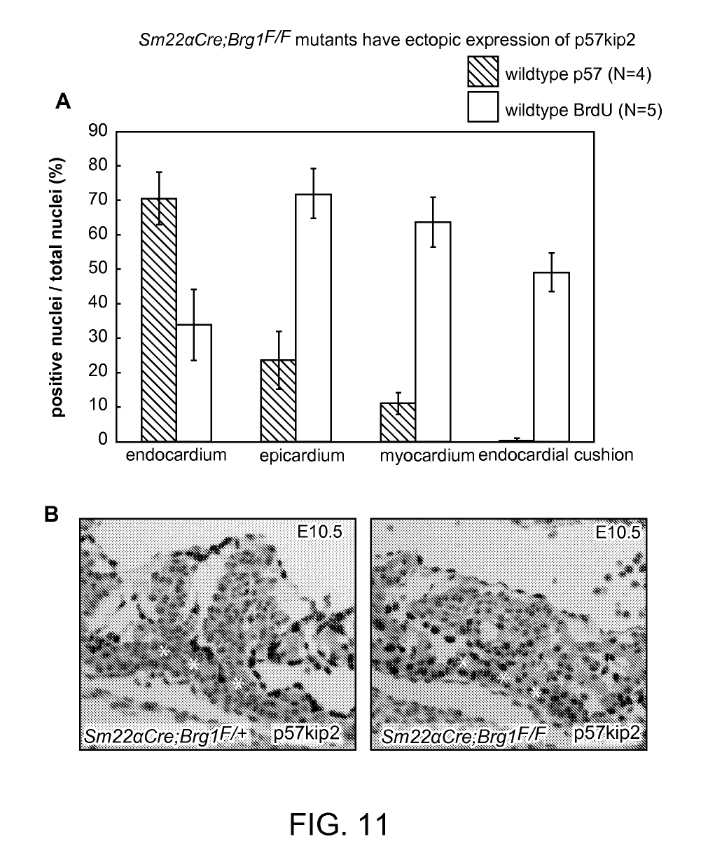

The BMP10 pathway is mis-regulated in the myocardium lacking Brg1 Failure of myocardial proliferation could reflect either a broad defect in muscle development or specific mis-expression of key regulators of cell division. Using RNA in situ hybridization, we surveyed the expression of key myocardial transcripts in Sm22αCre;Brg1^{F/F} embryos, including Nkx2.5, Gata4, MEF2C, Tbx3, Tbx5, CX43, Irx1, Irx2, NPPA, and BMP10. We found no significant changes in these transcripts in E11.5 Sm22αCre;Brg1^{F/F} hearts (FIG. 10) except for BMP10. BMP10 is required for the proliferation of myocardial cells, and BMP10 knockout embryos have thin myocardium. We found BMP10 expression was nearly abolished in the compact myocardium of Sm22αCre;Brg1^{F/F} starting at E10.5 (FIGS. 2N and 2O). We next examined the expression of p57^{kip2}, cyclin-dependent kinase (CDK) inhibitor whose myocardial expression is normally suppressed by Pax3 and BMP10. We found p57kip2 proteins were present in endocardial cells, but not myocardial or cushion mesenchymal cells in control embryos at E10.5 and E11.5 (FIG. 2P). Their presence correlated inversely with the BrdU incorporation rate of each cell type (FIG. 11A). However, in Sm22αCre;Brg1^{F/F} embryos, p57kip2 was ectopically expressed in the myocardium and at the site of septal primordium at E10.5 (FIGS. 2P and 2Q, and FIG. 11B), consistent with the reduction of BMP10 and early termination of cell proliferation in Brg1-null myocardium.

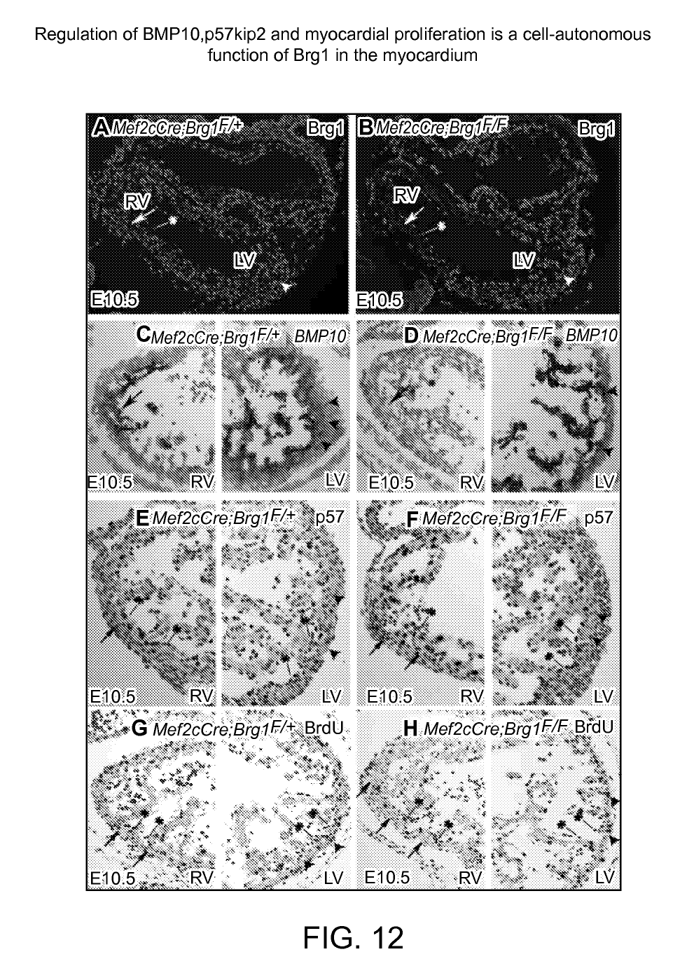

To rule out hemodynamic or other secondary causes of BMP10 and p57kip2 mis-regulation in Brg1-null myocardium, we used MEF2cCre mouse line to delete the Brg1 specifically in the right ventricle and outflow tract, leaving its expression intact in the left ventricle (FIGS. S5A and S5B). We found the Brg1-null right ventricle phenocopied the defects in Sm22αCre;Brg1^{F/F} ventricles, namely downregulation of BMP10 (FIGS. 12C and 12D), ectopic expression of p57kip2 (FIGS. 12E and 12F), and reduction of BrdU incorporation (FIGS. S5G and S5H); while the Brg1-positive left ventricle was normal, demonstrating a primary and cell-autonomous role of Brg1.

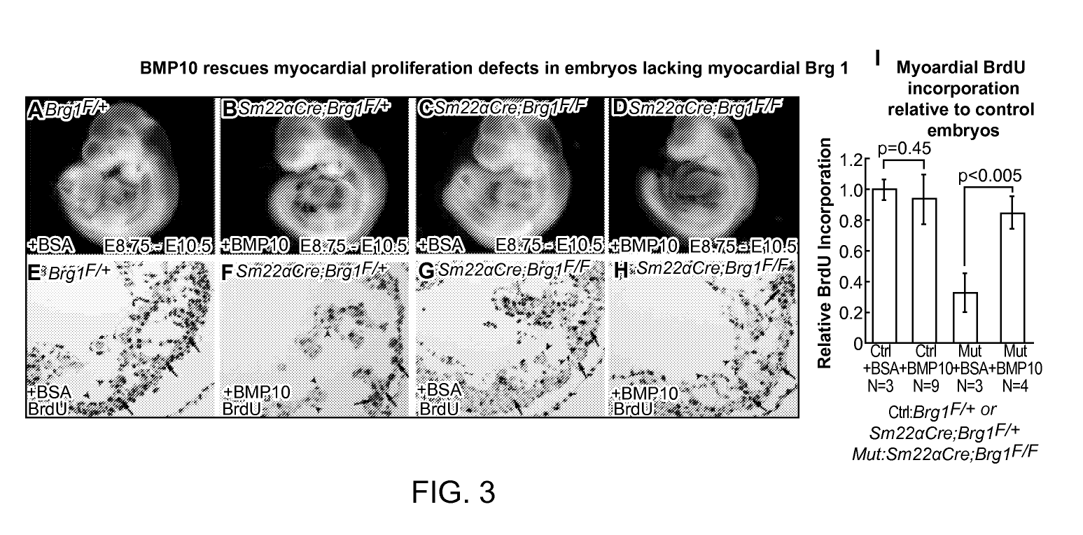

BMP10 deficiency causes myocardial proliferation defects in Sm22αCre;Brg1F/F embryos To test whether BMP10 deficiency limits myocardial growth in Sm22αCre;Brg1^{F/F} embryos, we performed pharmacological rescue experiments with whole embryo culture. Sm22αCre;Brg1^{F/F} and control embryos were cultured from E8.75 for 42 to 48 hours with or without BMP10 treatment. Cultured Sm22αCre;Brg1^{F/F} embryos were grossly comparable to littermate controls (FIGS. 3A-3D), but displayed myocardial proliferation defects indicated by a 67% reduction (100+/−7% in control vs 33+/−12% in mutant embryos) in BrdU incorporation (FIGS. 3A, 3C and 3I). Strikingly, BMP10 treatment increased BrdU incorporation in Sm22αCre;Brg1^{F/F} embryos by 2.6 folds and restored it to 85+/−10% (FIGS. 3F, 3H and 3I), which was not statistically different from control embryos treated with bovine serum albumin or BMP10. These findings indicate that BMP10 deficiency in Brg1-null myocardium is the cause of stunted myocardial growth.

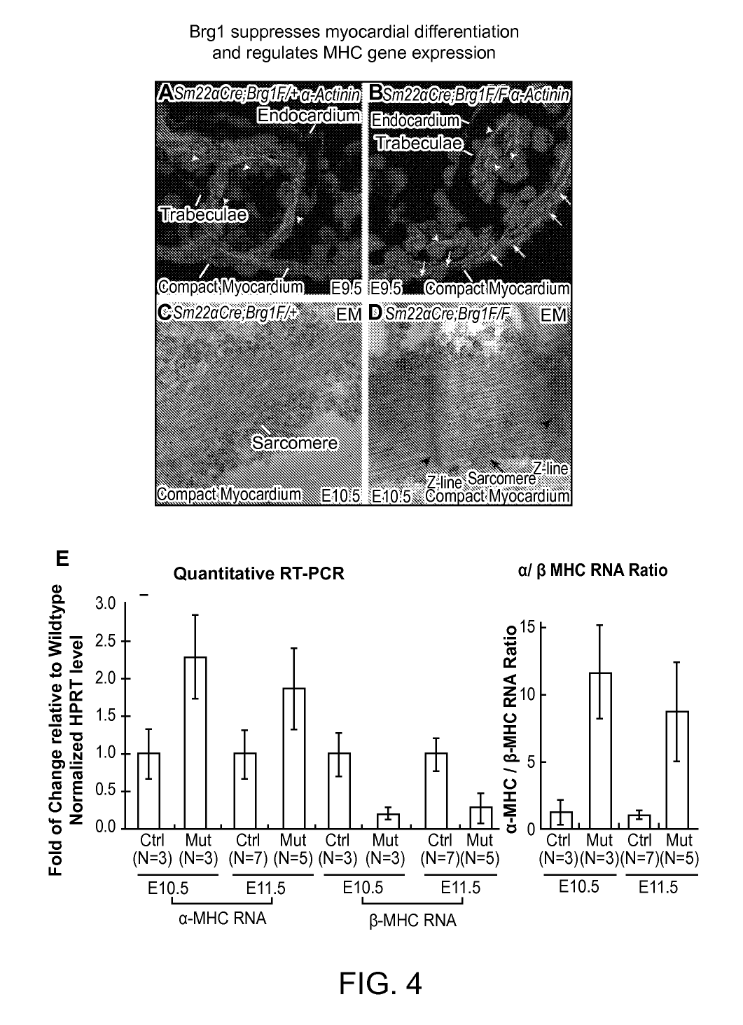

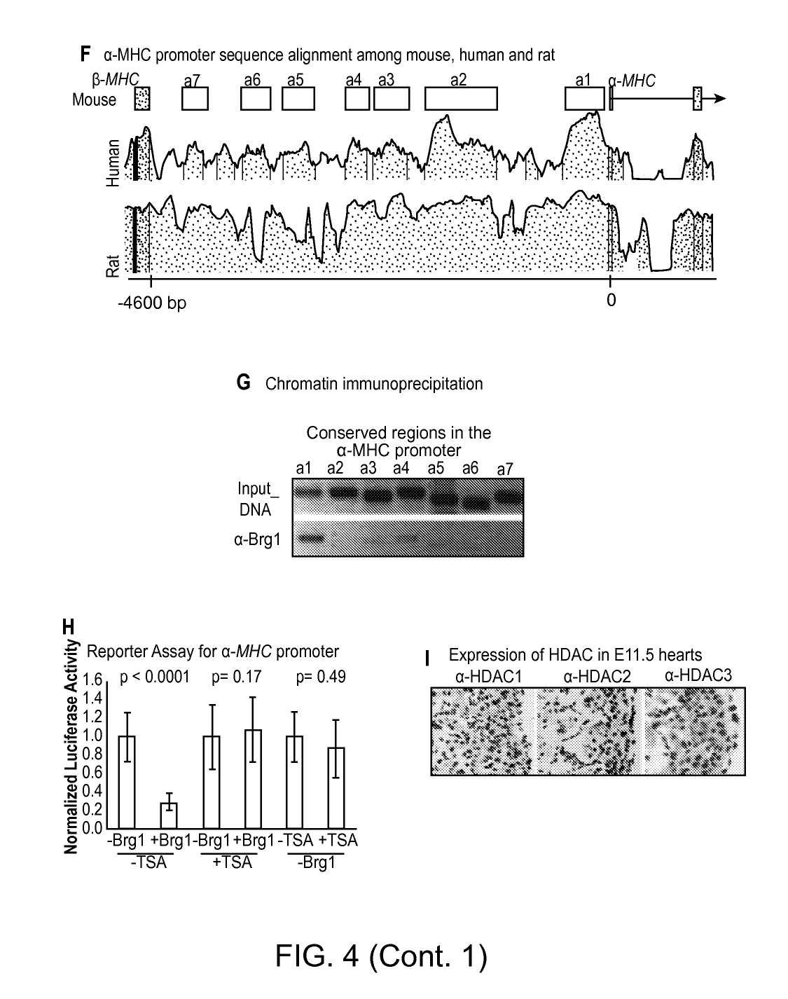

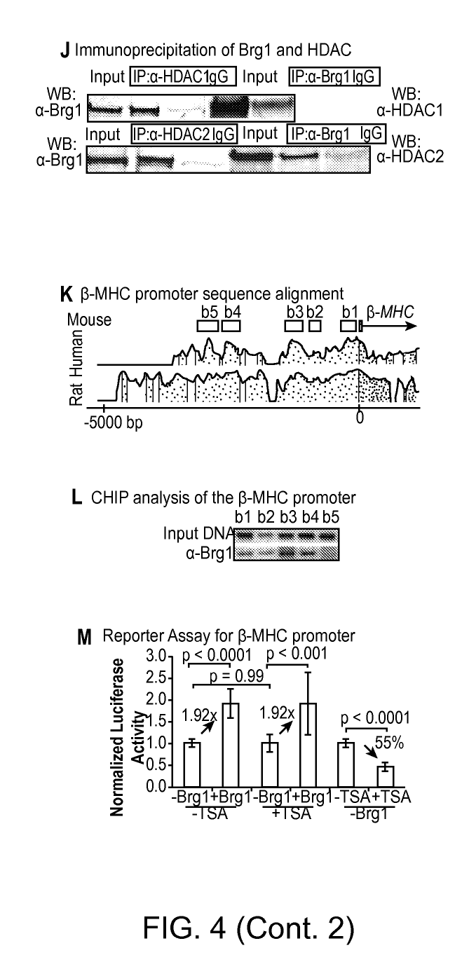

Myocardial cells lacking Brg1 undergo premature differentiation Since p57kip2 expression is associated with cell-cycle arrest and is required for proper differentiation of muscle cells, we examined if the early termination of myocardial proliferation in Sm22αCre;Brg1^{F/F} embryos was coupled with premature differentiation. By immunostaining, we found that α-actinin, a component of myofibril Z-lines, was organized into a segmented pattern in the compact myocardium of Sm22αCre;Brg1^{F/F} embryos, while remaining diffusely distributed in the controls at E9.5 (FIGS. 4A and 4B). Transmission electron microscopy studies showed that the control compact myocardium displayed short myofibril bundles scattered throughout the cytoplasm at E10.5 (FIG. 4C); while the Sm22Cre;Brg1^{F/F} myocardium contained mature myofibrils with consecutive sacromeres demarcated by Z-lines (FIG. 4D). Such advanced myofibril organization suggests that myocardial cells of Sm22Cre;Brg1^{F/F} have prematurely differentiated. To further assess the myocardial differentiation status of Sm22αCre;Brg1^{F/F} embryos, we quantitated the expression of different isoforms of myosin heavy chain, α-MHC and β-MHC, by RT-PCR on the total E10.5 and E11.5 ventricular RNA. We found that Sm22αCre;Brg1^{F/F} embryos highly expressed α-MHC, and down-regulated β-MHC (FIG. 4E, left panel), therefore increasing α-MHC/β-MHC transcript ratio by 10-12 folds (FIG. 4E, right panel). Given that α-MHC is predominantly expressed in the adult murine myocardium, and β-MHC mainly expressed in embryonic hearts, these findings indicate that Brg1-null myocardial cells are highly differentiated, indicating a role of BAF complexes in the suppression of myocardial differentiation.

The BAF complex represses the expression of α-MHC in a HDAC-dependent manner To test if the BAF complex directly regulate MHC expression in embryonic hearts, we checked Brg1 binding to MHC promoters The α-MHC and β-MHC genes are located next to each other with an approximately 4.6 Kb α-MHC upstream sequence in between (FIG. 4F). Using sequence alignment, we identified 7 highly evolutionarily conserved regions (a1-a7 in FIG. 4F) in the α-MHC promoter among human, rat and mouse. With approximately eighty E10.5 embryonic hearts per ChIP assay with an anti-Brg1 antibody, we found that of the 7 regions, the BAF complex was associated with the immediate promoter (a1 region) of α-MHC (FIG. 4G). To test the functional significance of this binding, we cloned different regions of the α-MHC promoter into pREP4, an episomal luciferase reporter vector that become chromatinized when transfected into mammalian cells. The reporter constructs were transfected into SW13 cells, which lack both Brg1 and its homolog Brm, and therefore lack functional BAF complexes, along with co-transfected Brg1-expressing or control plasmids. The reporter assays showed that restoring BAF complex function in SW13 cells caused a significant reduction (72+/−9%) in α-MHC reporter activity (FIG. 4H, 1st and 2nd columns), supporting a direct repression of α-MHC by the BAF complex.

In view that HDAC is a chromatin modifier that mediates transcriptional repression by limiting the accessibility of genetic loci, BAF complexes may require HDAC activity to repress α-MHC. To test this, we transfected SW13 cells with Brg1 expression vector and treated them with trichostatin A (TSA), and then measured the luciferase activity of α-MHC reporters. We found that inhibition of HDAC reversed the BAF-mediated repression of α-MHC in SW13 cells (FIG. 4H, 1st-4th column), indicating that Brg1 requires HDAC to repress α-MHC in the developing hearts. However, in the absence of Brg1, HDAC activity was not sufficient to repress the α-MHC promoter activity in SW13 cells (FIG. 4H, 5th and 6th columns), suggesting that the BAF proteins may be crucial for the recruitment of HDAC proteins to the immediate promoter site of α-MHC. These studies suggest that BAF may complex with HDAC proteins to regulate MHC expression.

We next investigated whether BAF proteins could physically complex with HDAC in embryonic hearts. By immunostaining, we first determined that Class I HDAC proteins, HDAC1, 2 and 3, were widely expressed at the E11.5 endocardium, myocardium, and epicardium (FIG. 4I). We then co-immunoprecipitated Brg1 and the three HDACs from E11.5 embryonic hearts. By western blot we found that Brg1 immunoprecipitates pulled down HDAC1, 2 and 3 proteins (FIG. 4J). Reciprocally, HDAC1 and HDAC2 immunoprecipitates pulled down Brg1 proteins (FIG. 4J). The anti-HDAC3 antibodies, however, did not allow immunoprecipitation studies. Taken together, these observations indicate that BAF and HDAC form co-repressor complexes in embryonic hearts to control α-MHC expression.

The BAF complex activates β-MHC expression independent of HDAC activity Using similar approaches, we analyzed the interaction of the BAF complex with the β-MHC promoter, where a 5.5 Kb upstream sequence is sufficient to direct a strong expression of β-MHC in cardiomyocytes. Within this sequence, we identified 5 highly conserved regions (b1-b5) among mouse, human and rat (FIG. 4K). Similarly, by ChIP analysis we observed that the BAF complex in embryonic hearts was widely associated with four of the five conserved regions of the β-MHC promoter (FIG. 4L). To test Brg1's activity on the β-MHC promoter, we transfected SW13 cells with Brg1-expressing or control plasmids and found that Brg1 enhanced the immediate promoter activity of β-MHC by 1.92 folds (FIG. 4M, 1st and 2nd columns). Interestingly, blocking HDAC activity with TSA had no significant impact on the Brg1-induced β-MHC promoter activation (FIG. 4M, 1st-4th columns), suggesting the BAF-mediated β-MHC activation does not require HDAC activity. In contrast, HDAC is necessary for the basal activity of β-MHC, since TSA treatment of SW13 cells resulted in 55% reduction of β-MHC promoter activity (FIG. 4M, 5th and 6th columns), indicating that HDAC has a BAF-independent role in the regulation of β-MHC expression. Overall, our biochemical studies and reporter assays support that the BAF complex and HDAC proteins co-repress the α-MHC expression, but have independent contributions to the activation of β-MHC expression.

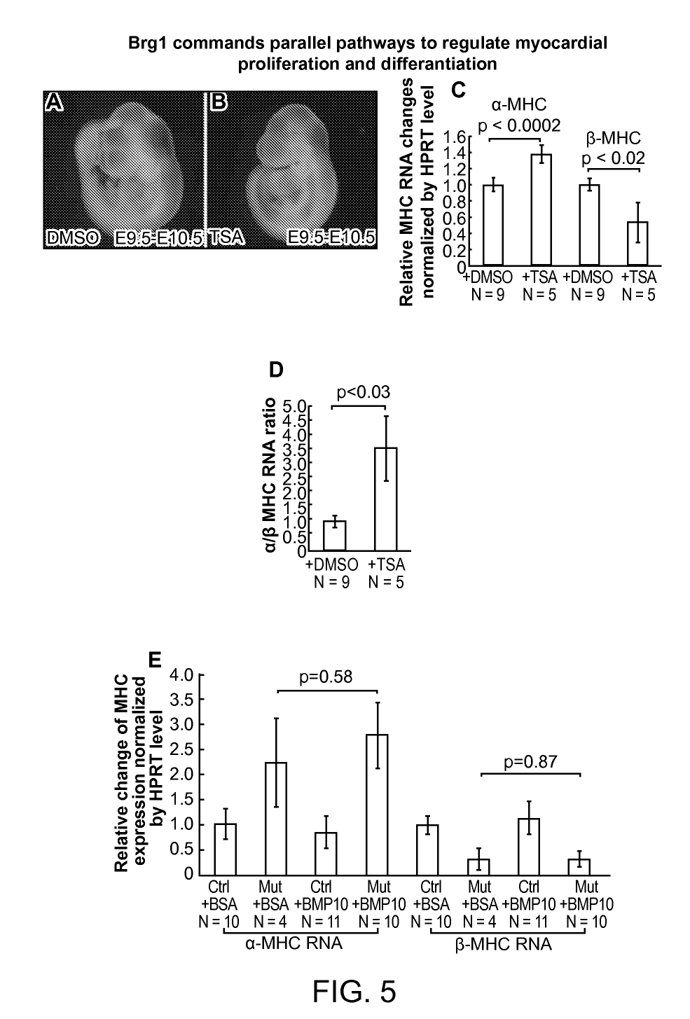

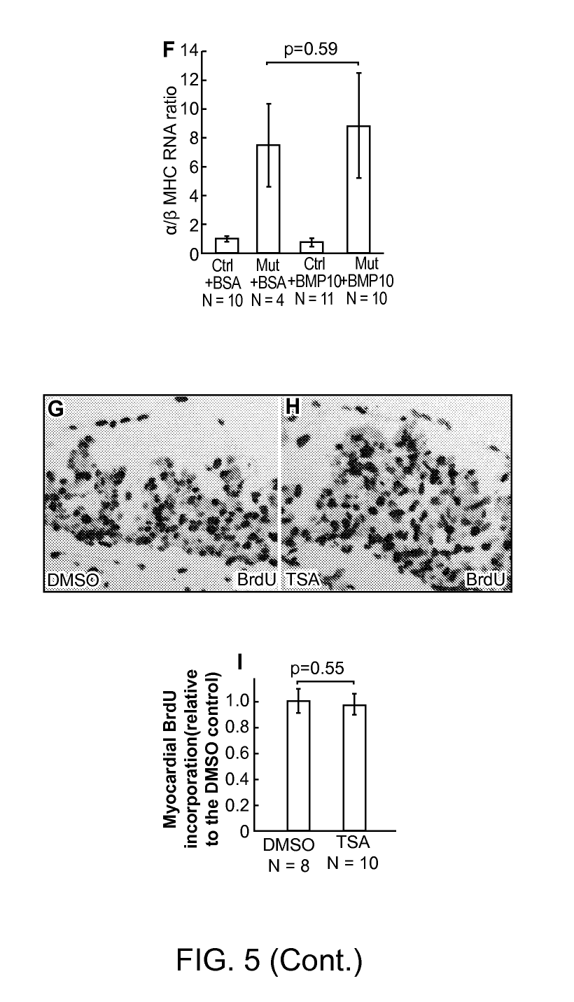

HDAC inhibition causes premature MHC switches in embryonic hearts Next we asked whether embryos deficient in HDAC activity would display premature α/β MHC switches as observed in Brg1-null myocardium. We cultured whole embryos from E9 to E10 with TSA or DMSO control, and analyzed ventricular expression of α-MHC and β-MHC. TSA treated embryos were grossly normal compared to littermates treated with DMSO (FIGS. 5A and 5B). However, by quantitative RT-PCR, we found that TSA-treated embryos significantly up-regulated α-MHC while down-regulated β-MHC, thereby inducing an increase in α/β MHC ratio by 3.5 folds (FIGS. 5C and 5D). Thus, embryos lacking HDAC activity displayed premature MHC switch as observed in embryos lacking Brg1, providing additional in vivo evidence supporting the physical and genetic interactions between BAF and HDAC proteins in the control of MHC expression of embryonic hearts.

Myocardial Brg1 controls the proliferation and differentiation of embryonic myocardial cells through independent but parallel pathways Since cell proliferation and differentiation are generally inversely regulated, it is possible that the premature MHC switches in Brg1-null myocardium could be partly secondary to the proliferative defects that trigger other BAF-independent regulatory mechanisms to promote cell differentiation. To decipher such BAF-independent cross-talks between cell proliferation and differentiation, we examined the MHC expression in cultured Sm22αCre;Brg1^{F/F} embryos whose myocardial proliferation defects had been rescued by BMP10 (FIG. 3). The direction and scale of α-MHC and β-MHC expression changes were quantitatively comparable between Sm22αCre;Brg1^{F/F} cultured in BSA and in utero embryos (compare FIGS. 5E and 4E), thereby validating the use of whole embryo culture to study myocardial MHC differentiation. Interestingly, although BMP10 treatment restored myocardial proliferation (FIG. 3), it failed to reverse the premature MHC differentiation of Sm22αCre;Brg1^{F/F} hearts. The BMP10-treated mutant embryos continued to over-express α-MHC and under-express β-MHC to a similar extent as the BSA-treated mutant embryos (FIGS. 5E and 5F), indicating that the BAF-BMP10 pathway does not govern MHC differentiation, and that the proliferation defects of Sm22αCre;Brg1^{F/F} myocardium per se do not cause premature MHC switches. On the other hand, we examined the proliferative state of myocardial cells in TSA-treated embryos that had premature MHC switches. We cultured wildtype embryos from E8.75 to E10.5 in TSA and treated them with BrdU for the last six hours of culture. Although TSA-treated embryos displayed premature MHC differentiation (FIGS. 5C and 5D), myocardial proliferation in these embryos was normal as indicated by comparable levels of BrdU incorporation between TSA- and DMSO-treated embryos (FIGS. 5G-I). Thus, the BAF-HDAC complexes do not control myocardial proliferation; and advanced MHC differentiation by itself is not sufficient to inhibit myocardial proliferation. Taken together, these studies suggest that the myocardial BAF complexes command two parallel pathways that independently control the proliferation and differentiation of myocardial cells in embryonic hearts.

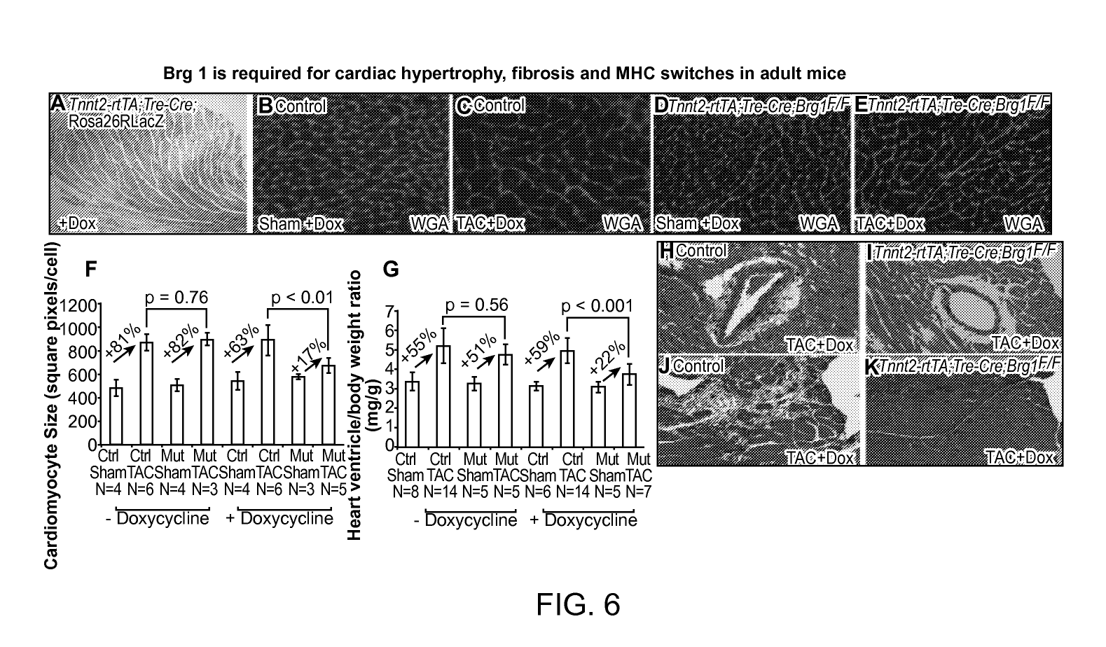

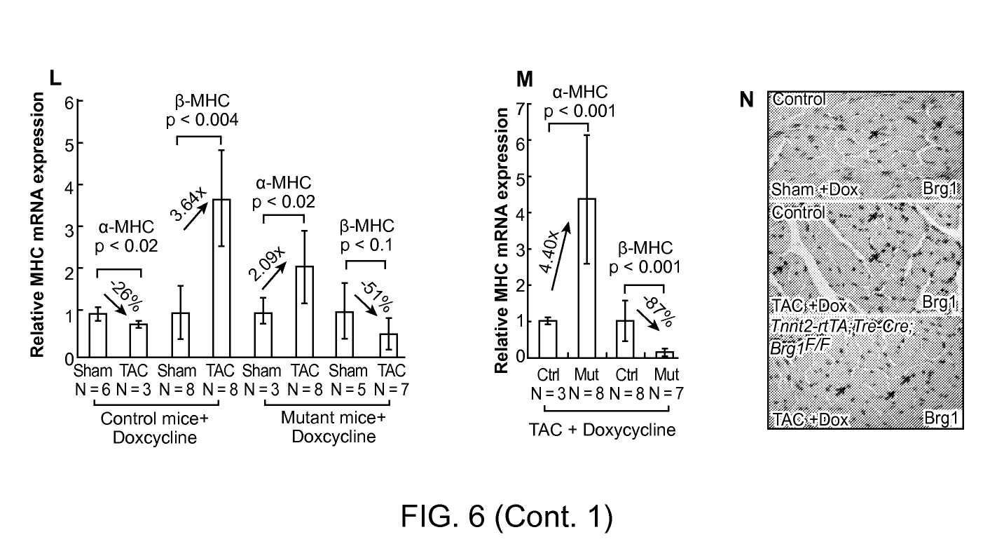

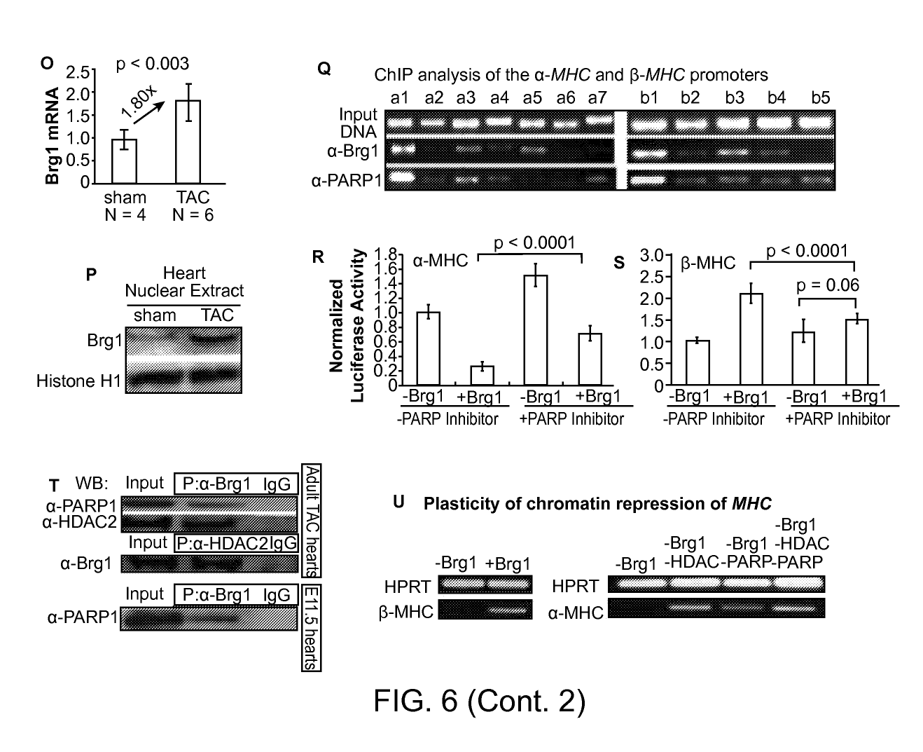

Brg1 is required for the hypertrophic growth and fibrosis of the heart Next, we asked if the BAF complex could also regulate cardiac growth and differentiation in adult hearts undergoing pathological remodeling. To bypass the embryonic lethality of Sm22αCre;Brg1^{F/F}, we used an inducible mouse transgenic line, Tnnt2-rtTA;Tre-Cre, that allows doxycycline-induced gene deletion specifically in adult myocardium. Feeding Tnnt2-rtTA;Tre-Cre adult mice with doxycycline-containing food pellets (6 gm doxycycline/kg of food, BioServ, Frenchtown, NJ) for 5 days was sufficient to activate the expression of a reporter gene, β-galactosidase, in myocardium (FIG. 6A).

We employed the transaortic constriction (TAC) technique (Hill et al., 2000) to pressure overload the heart and generate cardiac hypertrophy in control littermates and Tnnt2-rtTA;Tre-Cre;Brg1^{F/F} mice with or without doxycycline treatment. We verified the effectiveness of the TAC procedure four weeks later by performing echocardiographic analysis of the pressure gradient across the aortic valve. Only hearts of mice with pressure gradient >30 mmHg were harvested for analysis. We found that over the four-week period following surgery, the control mice developed severe cardiac hypertrophy with an increased cardiomyocyte size by 63+/−24% (FIGS. 6B, 6C, 6F), increased ventricular weight/body weight ratio by 59+/−21% (FIG. 6G), and significant perivascular and interstitial cardiac fibrosis (FIGS. 6H and 6J). Tnnt2-rtta-TreCre;Brg1^{F/F} mice fed with normal diet also displayed severe cardiac hypertrophy as the control mice in response to TAC (FIGS. 6F and 6G), indicating that the transgene itself had no effect on cardiac hypertrophy. Also, doxycycline treatment per se had no effects on the degree of TAC-induced changes of cardiomyocyte size or ventricular/body weight ratio (FIGS. 6F and 6G). In contrast, Tnnt2-rtTA;Tre-Cre;Brg1^{F/F} mice treated with doxycycline exhibited only mild cardiac hypertrophy in response to TAC. The cardiomyocyte size increased slightly by 17+/−11% (FIGS. 6D-6F); the ventricular weight/body weight ratio by 22+/−10% (FIG. 6G); and no ventricular fibrosis was observed (FIGS. 6I and 6K). Based on cardiomyocyte size and ventricular weight, there was an overall 63-73% reduction of cardiac hypertrophy in mice lacking myocardial Brg1. Thus, the BAF complex plays a critical role in the hypertrophic growth of the heart in response to pressure stress.

Brg1-null myocardium reverses the α- and β-MHC changes in response to cardiac stress We next investigated whether the BAF complex also regulated MHC expression in hypertrophic hearts. By quantitative RT-PCR, we found that control mice showed canonical changes of MHC expression characteristic of pathological hypertrophy in response to pressure overloading. The α-MHC expression decreased by 26% (control sham 100+/−21% vs control TAC 74+/−6%); while β-MHC increased by 3.6 folds (control 100+/−60% vs control TAC 364+/−109%) (FIG. 6L). In contrast, there was a failure of these MHC changes in mice lacking myocardial Brg1. Tnnt2-rtTA;Tre-Cre;Brg1^{F/F} mice that were treated with doxycycline and had undergone TAC showed a 2.1-fold increase of α-MHC expression (mutant sham 100+/−29% vs mutant TAC 209+/−85%) and a 51% reduction of β-MHC (mutant sham 100+/−72% vs mutant TAC 49+/−36%) (FIG. 6L). Overall, in response to pressure overload, the Brg1-null myocardium expressed 4.4-fold α-MHC and 0.13-fold β-MHC as much as the control myocardium (FIG. 6M). These reversed changes of MHC expression were not caused by reduced cardiac hypertrophy since the latter could only lessen, but not reverse, the canonical MHC changes of hypertrophic hearts. Therefore, the reversed MHC expression in Brg1-null myocardium is a direct consequence of the loss of Brg1/BAF transcriptional activity on the MHC promoters. Taken together, these data indicate that the BAF complex is required for pathological remodeling of diseased hearts, and to repress α-MHC and activate β-MHC expression in hypertrophic hearts.

Brg1 is reactivated by cardiac stress and complexes with PARP1 and HDAC proteins to regulate MHC expression While Brg1 is highly expressed in embryonic hearts, it is nearly turned off in adult myocardium with some endothelial expression (FIG. 6N, upper panel). Since Brg1 is required for cardiac hypertrophy, we speculated that Brg1 may be re-expressed in the diseased myocardium. Indeed, by immunohistochemistry, we observed elevated levels of Brg1 proteins in the myocardium within 7 days after the TAC procedure (FIG. 6N, middle panel), and Brg1-null myocardium showed no such response to TAC (FIG. 6N, lower panel). By quantitative RTPCR, we found that Brg1 expression in adult ventricles increased by 1.8 folds within 2 weeks after TAC (FIG. 6O). Western blot analysis showed that Brg1 proteins were easily detectable in the nuclear extracts of hearts after TAC, while they were minimal in normal hearts (FIG. 6P). These suggest that the reactivation of Brg1 expression by stress signals is a critical component of the hypertrophic process.

We next asked whether this reactivation of Brg1 could target the BAF complex to the promoters of MHC and regulate their expression in hypertrophic hearts. By ChIP analysis of TAC-treated hearts, we found that the BAF complex was highly enriched in the immediate promoters of both α-MHC and β-MHC (FIG. 6Q). Interestingly, this enrichment of BAF complex on MHC promoters occurred only in TAC-treated, but not sham-operated, hearts, consistent with Brg1 reactivation by the pressure stress. Also, the pattern of BAF association with MHC promoters in stressed hearts was strikingly similar to that in embryonic hearts (FIGS. 4G and 4L), indicating a common mechanism of BAF-mediated MHC expression control in embryonic and hypertrophic hearts.

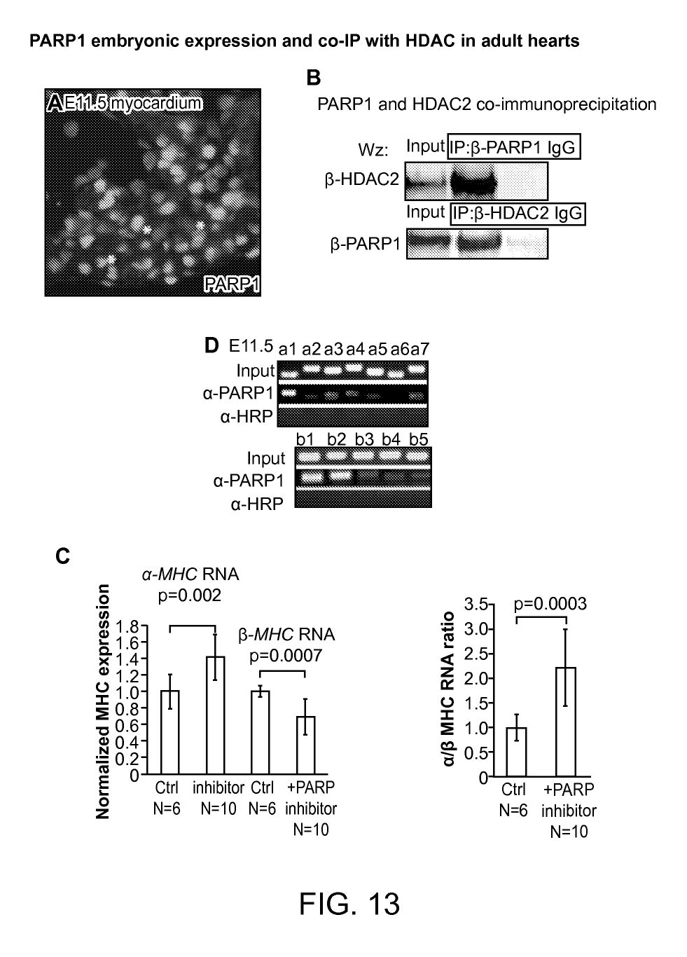

Besides HDAC proteins, poly (ADP-ribose) polymerase (PARP1) is the only other chromatin modifying enzyme known to play a role in the development of cardiac hypertrophy and MHC expression. However, whether PARP1 binds to the chromatinized MHC promoters are unknown. By ChIP analysis of wild type hearts subject to TAC, we found PARP1 protein was highly enriched in the immediate promoters of α-MHC and β-MHC in a pattern similar to that of the BAF complex (FIG. 6Q). Like Brg1, PARP1 associated with MHC immediate promoters only in TAC-treated, but not sham, hearts. Furthermore, we found that PARP1 cooperated with BAF to regulate α- and β-MHC expression since chemical inhibition of PARP1 activity by PJ-34 reduced both Brg1-mediated α-MHC repression and β-MHC activation in reporter assays using SW13 cells (FIGS. 6R and 6S). Next, we asked if these two proteins physically interact. We found PARP1 proteins co-immunoprecipitated with Brg1 in TAC-treated hearts as well as in E11.5 hearts (FIG. 6T). In addition, cultured embryos treated with PARP inhibitors (PJ-34) exhibited α- and β-MHC switches as observed in Brg1-null embryonic myocardium (FIG. 13C). Immunostaining and ChIP analyses of E11.5 hearts showed that PARP1 proteins were present in embryonic hearts and bound to the proximal promoters of α- and β-MHC, in a pattern similar to that of Brg1 (FIG. 13A, FIG. 13D). These data indicate that PARP1/BAF complexes to regulate MHC in both developing and stressed hearts. However, PARP1/BAF formed only minimally in sham-operated hearts, indicating that Brg1 reactivation by pressure stress is essential to trigger the formation of PARP1/BAF complexes.

Both Brg1 and PARP1 complexed with HDAC1 or HDAC2 in TAC-treated hearts (FIG. 6T and FIG. 13B). However, reporter studies indicate that Brg1 interacts with HDAC in α-MHC repression, but not β-MHC activation (FIG. 4M), consistent with the repressive roles of HDAC proteins. Together, these results suggest that BAF binds with PARP1 and HDAC proteins to form a chromatin-remodeling complex in the α-MHC immediate promoter to repress α-MHC expression; while BAF complexes with PARP1 in the β-MHC immediate promoter to activate β-MHC expression. Also importantly, the assembly of BAF/PARP1/HDAC complex to regulate MHC expression and cardiac hypertrophy depends on the reactivation of Brg1 by cardiac stress.

The plasticity of BAF-mediated chromatin repression of MHC in non-muscle SW13 cells. Given the interactions of BAF, HDAC and PARP1 on MHC reporters in SW13 cells, we next asked whether the BAF/PARP1/HDAC complex had any role in repressing endogenous MHC expression in these non-muscle cells that contain BAF subunits, but lack Brg1 and do not normally express MHC. We transfected these cells with Brg1-expressing plasmids and found that reconstitution of the BAF complex was sufficient to activate endogenous β-MHC expression in SW13 cells (FIG. 6U). Furthermore, inhibition of two components of the BAF/HDAC/PARP1 complex de-repressed endogenous α-MHC expression in SW13 cells (FIG. 6U). These observations validated the use of heterologous SW13 cells to study BAF's regulation of MHC since all factors essential for MHC regulation were present or activated by the BAF complex. Also, the BAF/HDAC/PARP-mediated chromatin repression of MHC can be reversed by modulating BAF, HDAC and PARP activity, indicating therapeutic potentials in modifying MHC changes for patients with cardiac hypertrophy. This prompted us to investigate whether Brg1 contributes to the development of human hypertrophic cardiomyopathy.

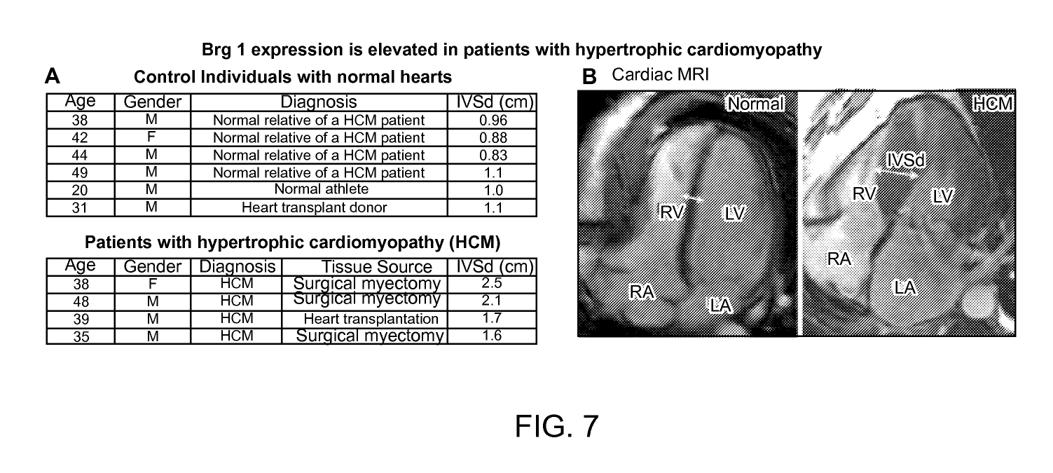

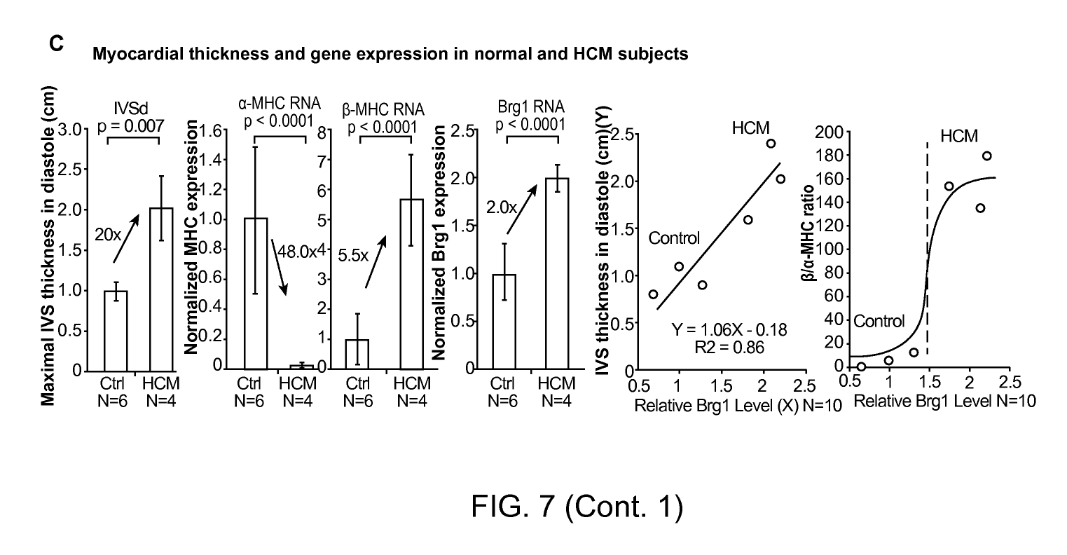

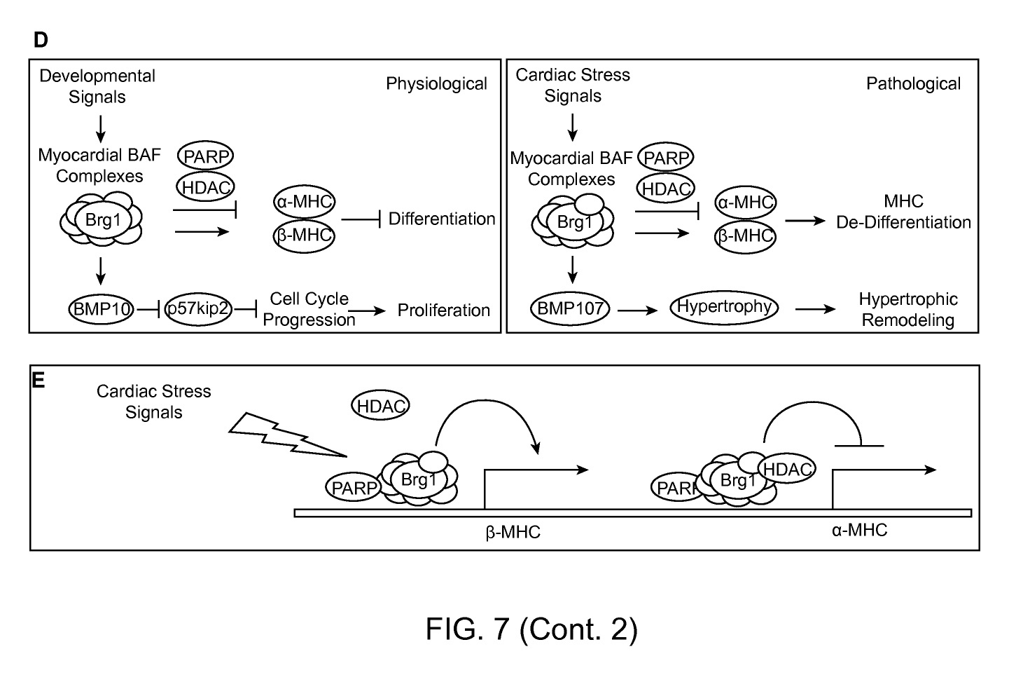

Reactivation of Brg1 in patients with hypertrophic cardiomyopathy Human hypertrophic cardiomyopathy (HCM) is a disease characterized by left ventricular hypertrophy and a non-dilated cardiac chamber without obvious causes such as hypertension or aortic stenosis. We first compared a set of normal subjects, including relatives of HCM patients, an athlete and a heart transplant donor, with a group of patients with diagnosed HCM (FIG. 7A). These two groups of individuals were approximately age-matched (37.3+/−10.4 vs 40+/−5.6, p=0.66), and predominantly male. Cardiac magnetic resonance imaging (MRI) was used to visualize the heart and myocardial thickness of normal and HCM subjects. The maximal thickness of interventricular septal myocardium during diastole (IVSd) was measured to quantitate the severity of HCM (FIG. 7B). As measured by MRI, the IVSd in the HCM group was 2.02 folds as thick as in the control group (0.98+/−0.11 vs 1.98+/−0.41, p<0.007) (FIG. 7C). We next examined MHC and Brg1 expression in heart tissues obtained from six heart transplant donors and four HCM patients who received surgical myectomy or heart transplantation. By quantitative RT-PCR, we observed in HCM hearts a 48-fold reduction of α-MHC, a 5.5-fold increase of β-MHC and a 2-fold increase of Brg1 expression. The loss of α-MHC, gain of β-MHC and activation of Brg1 resembled the changes seen in mice with TAC-induced hypertrophy, suggesting a similar pathogenic role of Brg1 in HCM patients. Indeed, Brg1 expression correlated linearly with maximal IVSd among HCM and donor hearts (linear regression R2=0.86). Furthermore, a threshold of 1.5-fold Brg1 elevation predicts HCM and adverse β/α-MHC changes (FIG. 7C). These studies demonstrate that Brg1/BAF contribute to the development of human cardiomyopathy, providing a therapeutic target for reversing pathological changes in human disease.

Our studies demonstrate similar mechanisms directed by the BAF complex in the control of the balance between myocardial proliferation and differentiation in embryonic hearts as well as the hypertrophic growth of adult hearts under pathological stress (FIG. 7D). In embryos, the BAF complexes maintain the cardiomyocytes in a proliferative state by sustaining BMP10, and inhibit their differentiation by directly controlling the expression of cardiac MHC genes with HDACs and PARP1 (FIG. 7D, left panel). Likewise, in adults the BAF complexes is required for pathological hypertrophic growth and controls the expression of MHC with the same embryonic partners (FIG. 7D, right panel). BMP10 may be involved in the BAF-dependent cardiac hypertrophy. Furthermore, the assembly of BAF/HDAC/PARP1 complexes on MHC promoters only takes place under cardiac stress (FIG. 7E). Because Brg1 is important in keeping the embryonic cardiomyocytes in a proliferative progenitor state, and is required for pathological remodeling, the BAF complex has regenerative and therapeutic implications.

The BAF complex determines the cell fates of developing myocardial cells Proliferation and differentiation are two important cellular events that are generally inversely regulated. We used a combination of mouse genetics, whole embryo cultures, and molecular biology/biochemistry to dissect out the cross-interactions between the two processes, and reveal independent but coordinated control mechanisms for each. We show that BAF complexes separately control these two cellular events through distinct pathways of BMP10 and HDACs in developing embryos. There appears no further BAF-independent cross-talking between myocardial proliferation and MHC differentiation based on the following observations: BMP10 rescues proliferation without normalizing differentiation in Brg1-null myocardium; conversely, the loss of HDAC activity triggers myocardial cells to express mature MHC isoform but does not affect their proliferation. Therefore, the proliferation and differentiation defects observed in Brg1-null myocardium are the direct results of BAF inactivation rather than secondary defects derived from one another. These findings indicate the existence of a high-ranking regulator, BAF, which initiates parallel paths to determine the cellular fates of developing myocardial cells.

In the light of previous research on BAF's roles in promoting differentiation, it is surprising that in the embryonic myocardium, the BAF complexes actively inhibit this process instead. The Brg1-null embryonic cardiomyocytes have early cell cycle arrest and are prematurely differentiated. Two possibilities explain this phenomenon. First, BAF complexes could have antagonistic roles in differentiation according to tissue or cell type. In fact, based on recent findings of combinatorial assembly of BAF complexes with unique subunit composition, it is likely that there exist embryonic cardiomyocyte-specific BAF complexes with distinct functions. Second, BAF complexes may have different functions depending on the timing or progression within a cell lineage. For example, BAF complexes largely promote further differentiation of cells that are already specified for certain lineages, such as in T-cell development, or differentiating mesodermal cells to cardiac lineage. However, they are also required for self-renewal and pluripotency of embryonic stem cells. The spectrum of genes affected by BAF mutations appears to depend on the timing when BAF is disrupted; with its target specificity becomes narrower in more differentiated cells. Therefore, the BAF complex may have temporal- and spatial-specific functions dictated by its dynamic composition. The understanding of how BAF balances the growth and differentiation of cardiac cells provides a basis for expanding and maturing cardiomyocytes derived from ES or iPS cells for regenerative purpose.

BAF forms physical complexes with HDAC and PARP1 to control MHC expression. Recent works have shown that in addition to specific signaling pathways and transcription factors, chromatin remodeling play an important role in the hypertrophy process. Histone deacetylases (HDACs) and poly (ADP-ribose) polymerase (PARP) are the two classes of chromatin-modifying enzymes currently known to regulate cardiac hypertrophy. Pharmacologic inhibition of HDACs and PARP1 has been shown to decrease hypertrophy and also to reduce myocardial apoptosis following infarction. Furthermore, genetic studies have shown that class I HDACs and PARP1 knockout animals are resistant to many hypertrophic stimuli. Here, we identified Brg1 as a new chromatin remodeller important in the hypertrophic process. And intriguingly, these three classes of remodellers act together to specifically control the expression of MHC genes; they cooperate with one another and bind to similar regions in the MHC promoters. This indicates that regardless of the hypertrophic stimuli and the specific pathways triggered, the chromatin may ultimately be where all the signals converge for the regulation of MHC genes and possibly other structural genes to trigger the pathological remodeling of stressed hearts.

The embryonic expression of MHC isoforms is also controlled by BAF, HDAC and possibly PARP1. Both HDAC1/2/3 and PARP1 proteins are abundant in embryonic myocardial nuclei, and form physical complexes with Brg1 to regulate MHC. In addition, the interactions of these chromatin remodeling enzymes are essential in suppressing MHC expression in SW13 cells, human adrenal carcinoma cells that lack Brg1. These non-muscle SW13 cells do not normally express MHC. However, endogenous β-MHC expression in these cells can be induced by reconstituting the BAF complex. Also surprisingly, α-MHC is deprepressed by inactivating two components of the BAF/HDAC/PARP complex in these cells. These findings suggest a critical role of the BAF/HDAC/PARP complex in “locking” MHC in non-muscle cells, and the plasticity of this regulatory process. Therefore, biochemical interactions of these chromatin remodellers provide a mechanism for the opposite regulation of α- and β-MHC expression in developmental and pathological conditions. Indeed, BAF complexes and thyroid hormone receptors appear to be the only direct mechanisms currently known to antithetically regulate α- and β-MHC immediate promoters.

Further investigations are needed to fully understand how these complexes are assembled to modify the chromatin environment of MHC promoters under different pathophysiological conditions. Our studies suggest that BAF may play a central role in assembling the trimeric complex on the α-MHC promoter in hypertrophic hearts based on the following: First, Brg1 is shut down in adult cardiomyocytes, but reactivated by cardiac stresses. Second, Brg1-HDAC or Brg1-PARP complex could be effectively pulled down only after the heart has been pressure overloaded and Brg1 reactivated. Third, ChIP analysis shows that PARP1 does not bind to the immediate promoter of α-MHC until Brg1 is activated by cardiac stresses. PARP1 then binds to the promoters in a pattern similar to Brg1's. The trimeric complex on the MHC promoter may then recruit or interact with transcription factors such as TRα1, TRβ31, TEF1, MEF2, SRF, GATA4 and NFAT to regulate MHC expression in hypertrophic hearts.

Furthermore, considering HDACs and PARP1 are enzymes that covalently modify histones, these two components of the complex may mark the histones and help anchor BAF to these sites of MHC promoter since BAF subunits such as DPF3/BAF45c can read modified histones. Other intriguing questions are whether BAF components could be modified by acetylation/deacetylation and poly-ADP-ribosylation by HDACs and PARP1, how such modifications affect BAF's protein stability and its interaction with chromatin or other pathophysiological MHC regulators such as miR208 and thyroid hormone. Also, it remains to be elucidated how BAF interacts with different Class I and II HDAC proteins that have seemingly antagonistic effects on cardiac hypertrophy, and how combinatorial assembly of BAF with PARP and HDAC family members dictate their chromatin recognition and target specificity.

Implication for human hypertrophic heart disease Although adult human hearts predominantly express β-MHC, the disappearance of α-MHC and the expression of even more β-MHC in human cardiomyopathy directly translate to reduced cardiac contractility and ultimately worsened clinical outcome. Therefore, restoring this balance of MHC isoforms remains of great interest to revert pathological progression of hypertrophic hearts. Our studies show that Brg1 is not expressed in healthy adult myocardium, but is activated in the diseased heart to regulate MHC expression in mice. Preventing its re-expression in mice averts pathological MHC shift, lessens hypertrophy and abolishes cardiac fibrosis. As in murine myocardium, Brg1 proteins are not detectable in normal human adult myocardium, but Brg1is activated in patients with hypertrophic cardiomyopathy. Its level correlates with disease severity and associated MHC changes. A threshold of Brg1 elevation separates normal subjects from patients with severe disease, demonstrating Brg1 contributes to the development of human cardiomyopathy. Therefore, Brg1/BAF complexes provide a therapeutic target for hypertrophic heart disease. Because Brg1 is activated only by disease, anti-Brg1 drugs will affect only diseased hearts with few side effects.

### EXPERIMENTAL PROCEDURES

Mice. Sm22αCre, Brg1^{F/F}, Mef2cCre, R26R and Tnnt2-rtTA;Tre-Cre mice have been described previously (Boucher et al., 2003; Soriano, 1999; Stankunas et al., 2008; Sumi-Ichinose et al., 1997; Verzi et al., 2005). Embryonic age was determined by conventional postcoital dates and confirmed by ultrasonography. The use of mice for studies is in compliance with the regulations of Stanford University and National Institute of Health.

Chromatin Immunoprecipitation Chromatin was isolated as previously described, with modifications for primary embryonic and adult hearts and primers.

Cloning and Luciferase Reporter Assay Full length as well as serially truncated versions of intergenic α-MHC and β-MHC promoters were cloned into pREP4-Luc reporter plasmid, as detailed in the supplementary method. The constructs were transfected into SW13 cells with lipofectamine 2000 (Invitrogen, Calif.) along with pREP7-RL as a transfection efficiency control and Brg1 expression vector with the appropriate empty vector control. Luciferase activity was measured and normalized to that of Renilla luciferase construct using the Dual-Luciferase Reporter System (Promega, Wis.).

Human heart tissue collection Human heart tissue from patients with HCM was acquired at the time of cardiac transplantation or surgical myectomy. 3-8 g of tissue was removed from the proximal septum. Normal tissue was acquired from heart transplant donors where the heart was not used for transplantation or by biopsy immediately following implantation. Heart tissue was flash frozen in liquid nitrogen immediately and stored at −80° C. The use of human tissues is in compliance with Stanford University regulation.

Histology, RNA in situ hybridization and Immunostaining Histological analysis, immunostaining and RNA in situ hybridization were performed as described. All these procedures were performed on 7 μm paraffin sections of the heart. Hematoxylin and eosin (H&E) stain was performed according to standard protocols. The probes used for the RNA in situ hybridization were described in the text. The following primary antibodies were used for immunostaining: G7 Brg1 (Santa Cruz Biotechnology, Calif.), p57kip2 (Lab Vision, Fremont, Calif.), α-actinin (Sigma-Aldrich, St. Louis, Mo.), and troponin T (Hybridoma Bank, University of Iowa). Primary antibodies were detected either directly by fluorescent anti-mouse secondary antibodies or following manufacturer's protocol using ABC kit (Vector Labs, Burlingame, Calif.) with hematoxylin counterstaining.

Quantitative analysis of myocardial development H&E-stained 7 μm paraffin sections were used for the morphometric analysis of the compact and trabecular myocardium. The following parameters were measured: thickness of compact 4 myocardium, number of trabeculi directly connected to compact myocardium, and trabecular area normalized to ventricular size. BrdU-stained sections were used to count the number of BrdU-positive cells per area of myocardium or per number of cells in the endocardium or endocardial cushions. All quantification and scale measurement were performed using NIS-Elements software (Nikon).

Whole embryo culture Embryos (E875) with intact yolk sacs were isolated and incubated in 1 ml culture medium containing 97% rat serum (Harlan Biosciences, Cincinnati, Ohio), 2 mg glucose, 100 U penicillin G and 100 μg streptomycin that was rotated slowly at 3700 and gassed periodically (every 8 hours) with 20% oxygen, 5% carbon dioxide, 75% nitrogen from E8.75 to E9.5, and 70% oxygen, 5% carbon dioxide and 25% nitrogen from E9.5 to E10.5. BMP10 (10 nM, RD Systems, Minneapolis, Minn.), BSA (125 ng/ml), TSA (100-500 nM, Sigma-Aldrich, St. Louis, Mo.), PJ-34 (20 uM, Alexis Corporation, San Diego)), BrdU (30_g/mL, Simga-Aldrich) or DMSO was added directly to the culture medium or during the last six hours for BrdU. Embryos were harvested 36-48 hours later for analysis.

Immunostaining. The following primary antibodies were used for immunostaining: G7 Brg1 (Santa Cruz Biotechnology, Calif.), p57kip2 (Lab Vision, Fremont, Calif.), α-actinin (Sigma-Aldrich, St. Louis, Mo.), and troponin T (Hybridoma Bank, University of Iowa). Primary antibodies were detected either directly by fluorescent anti-mouse secondary antibodies or following manufacturer's protocol using ABC kit (Vector Labs, Burlingame, Calif.) with hematoxylin counterstaining.

Proliferation and apoptosis analysis. Pregnant mice were injected with BrdU (Sigma, 100 μg/ml, intraperitoneal injection) for 6 hours prior to embryo isolation at E10.5 or E11.5. Incorporated BrdU was stained in the tissue-section of the heart according to manufacturer's protocol (Zymed Laboratories, South San Francisco, Calif.). Apoptosis of embryos were analyzed by TUNEL staining using a kit (Roche, Applied Science, Indianapolis, Ind.).

Quantitative RT-PCR analysis. Quantitative RT-PCR analyses were performed as described previously to examine gene expressions in the cardiac ventricles of E10.5/E11.5 embryonic hearts and adult hearts. The following primer sequences were used: Murine MHO, 5′ primer ACGGTGACCATAAAGGAGGA, 3′ primer TGTCCTCGATCTTGTCGAAC. Murine β-MHC, 5″ primer GCCCTTTGACCTCAAGAAAG, 3′ primer CTTCACAGTCACCGTCTTGC. Murine HPRT, 5′ primer GCTGGTGAAAAGGACCTCT, 3′ primer CACAGGACTAGAACACCTGC. Murine Brg1, 5″ primer CACCTAACCTCACCAAGAAGATGA, 3′ primer CTTCTTGAAGTCCACAGGCTTTC. Human H3F3A, 5′ primer AAAACAGATCTGCGCTTCCA, 3′ primer TTGTTACACGTTTGGCATGG. Human Brg1, 5′ primer AGTGCTGCTGTTCTGCCAAAT, 3′ primer GGCTCGTTGAAGGTTTTCAG. Human α-MHC and β-MHC were by Taqman probes (Applied Biosciences, Foster City, Calif.. RT-PCR reactions were performed using SYBR green master mix (BioRad, Hercules, Calif.) or Taqman reagents (Applied Biosciences), and the primer sets were tested to be quantitative. Threshold cycles and melting curve measurements were performed with software.

Chromatin Immunoprecipitation Hearts from approximately eight litters of E10.5-E11.5 Swiss-Webster mice were dissected in chilled PBS, and subsequently fixed with 1% PFA and washed with 0.125M glycine. Adult hearts were minced before fixing with PFA. Cells were lysed by cell lysis buffer (10 mM Hepes pH 7.5, 85 mM KCl, 0.5% NP-40, protease inhibitor (#78410, Pierce, Rockford, Ill.). Nuclei were isolated by disruption using a B Bounce, and washed with SDS lysis buffer (1% SDS, 10 mM EDTA and 50 mM Tris, pH 8.1). Chromatin was sonicated to generate average fragment sizes of 100-200 bp, and immunoprecipitated using anti-Brg J1 antibody (Wang et al., 1996), anti-PARP1 antibody (N-20, sc-1561, Santa Cruz Biotechnology, Santa Cruz, Calif.) and anti-HRP control. Isolation of immunoprecipitated chromatin was done according to manufacturer's protocol (Upstate). FOR primers (a1-a7, b1-b5) were designed to amplify the following regions in the α-MHC (a1-a7) and 3-MHC (b1-b5) promoters: a1 (−357 to −463); a2 (−1092 to −1237); a3 (−1775 to −1908); a4 (−2141 to −2290); a5 (−2997 to −3121); a6 (−3378 to −3486); a7 (−3569 to −3714); b1 (−64 to −205); b2 (−912 to −1061); b3 (−1374 to −1518); b4 (−2284 to −2409); b5 (−2690 to −2827). The DNA positions are denoted relative to the transcriptional start site (+1).

Cloning and Luciferase Reporter Assay Specific truncations for the reporter constructs are as follows: for α-MHC promoter, full length promoter spans from 4243 by upstream and 192 by downstream of the transcription start site (−4243, +192), serially truncated versions span (−2537, +192), (−1802, +192), and (−462, +192). Likewise, β-MHC promoters span (−3561, +222), (−1770, +222), and (−835, +222).

Co-Immuunoprecipitation Embryonic hearts (E10.5) were homogenized in cold NP-40 lysis buffer (25 mM K-HEPES pH 7.5, 250 mM KCl, 12.5 mM MgCl2, 0.5% NP-40, 8% glycerol, 1 mM DTT, protease and phosphatase inhibitors cocktail) using a tissue homogenizer. The lysates were then cleared by centrifugation and protein concentration was determined by Bradford. Pre-clearing of the lysates was performed with protein A/G beads (Pierce, Thermo Scientific, Rockford, Ill.). After removing the A/G beads by centrifugation, the lysates were incubated with 1 μl primary antibody (Brg1 (H88) Santa Cruz Biotechnology, Santa Cruz, Calif.; HDAC1(ab7028) Abeam Cambridge, Mass.; HDAC2 (H54) Santa Cruz Biotechnology, Santa Cruz, Calif.; HDAC3 (2632, Cell Signaling, Danvers, Mass.); anti-PARP1 antibody (N-20, sc-1561, Santa Cruz Biotechnology, Santa Cruz, Calif.) with rotation at 4° C., overnight. Lysates were now incubated with Protein NG beads to precipitate complexes, for 2 hours at 4° C. with rotation. The immunocomplexes with protein NG beads were now recovered by centrifugation and washed twice with lysis buffer. SDS buffer was added to the precipitate and boiled for 10 min. Proteins were size separated in SDS-PAGE. The gels were blotted onto an lmmobilon-P membrane (Millipore, Bedford, Mass.), blocked with 5% non-fat dry milk and incubated with the previously described antibodies. HRP-conjugated secondary antibodies (Jackson ImmunoResearch Laboratories, West Grove, Pa.) were used for detection using ECL method (GE Healthcare Bio-Sciences Corp Piscataway, N.J.).

Transaortic Constriction (TAC). Mice were fed with doxycycline food five days prior to TAC operation to induce deletion of Brg1. Surgeries were adapted from (Trivedi et al., 2007) and were performed on adult mice of 11-12 weeks of age and between 25 and 30 grams of weight. Mice were fed with doxycycline food pellets (6 gm doxycycline/kg of food, Bioserv, Frenchtown, N.J.) five days prior to the TAC operation. Mice were anesthetized with ketamin (40 mg/kg, ip), xylazine (10 mg/kg, ip) and isoflurane (2-3%, inhalation). Mice were then intubated with a 20-gauge intravenous catheter and ventilated with a mouse ventilator (model Minivent, Harvard Apparatus, Inc). Anesthesia was maintained with inhaled isoflurane (1-2%). A longitudinal 5-mm incision of the skin was made with scissors at midline of sternum. The chest cavity was opened by a small incision at the level of the second intercostal space 2-3 mm from the left sternal border. While opening the chest wall, the chest retractor was gently inserted to spread the wound 4-5 mm in width. The transverse portion of the aorta was bluntly dissected with curved forceps. Then, 6-0 silk was brought underneath the transverse aorta between the left common carotid artery and the brachiocephalic trunk. One 26-gauge needle was placed directly above and parallel to the aorta. The loop was then tied around the aorta and needle, and secured with a second knot. The needle was immediately removed to create a lumen with a fixed stenotic diameter. The chest cavity was closed by 6-0 silk suture. Sham-operated mice underwent similar surgical procedures, including isolation of the aorta, looping of aorta, but without tying of the suture.

Morphometric Analysis of Cardiomyocytes Paraffin sections of the heart were immunostained with a fluoresecin isothiocyanate-conjugated Wheat Germ Agglutinin (WGA) antibody (F49, Biomeda, Foster City, Calif.) that highlighted the cell membrane of cardiomyocytes. Cell areas outlined by WGA staining were determined by the number of pixels enclosed using the NIS element software (Nikon). Approximately 250 cardiomyocytes of the papillary muscle at the mid left ventricular cavity were measured to determine the size distribution, p-values were calculated by the Student-t test.

Western Immunoblot Analysis Whole hearts were collected and washed once with ice-cold phosphate-buffered saline (PBS) and homogenized with homogenizer (Fisher Scientific, Power gen 125) in buffer A (25 mM Hepes, pH 7.0, 25 mM KCl, 5 mM MgCl₂, 0.05 mM EDTA, 10% glycerol, 0.1% NP-40) for 1 min. The homogenates were centrifuged twice at 750 g for 10 min at 4° C. The nuclear pellets were resuspended in buffer B (50 mM Tris-Hcl, pH 6.8, 2% SDS, 100 mM DTT, 10% glycerol). After boiling for 10 min and centrifugation at 12,000 rpm for 10 min, the supernatants were collected and frozen at −80° C. The blots were reacted with antibodies for Brg1 (Santa Cruz, sc-17796) and Histone H1 (Santa Cruz, sc-34464), followed by horseradish peroxidase (HRP)-conjugated antimouse IgG or HRP-conjugated anti-goat IgG (Jackson). Chemiluminescence was detected with ECL Western blot detection kits (GE) according to the supplier's recommendations.

### Example 2

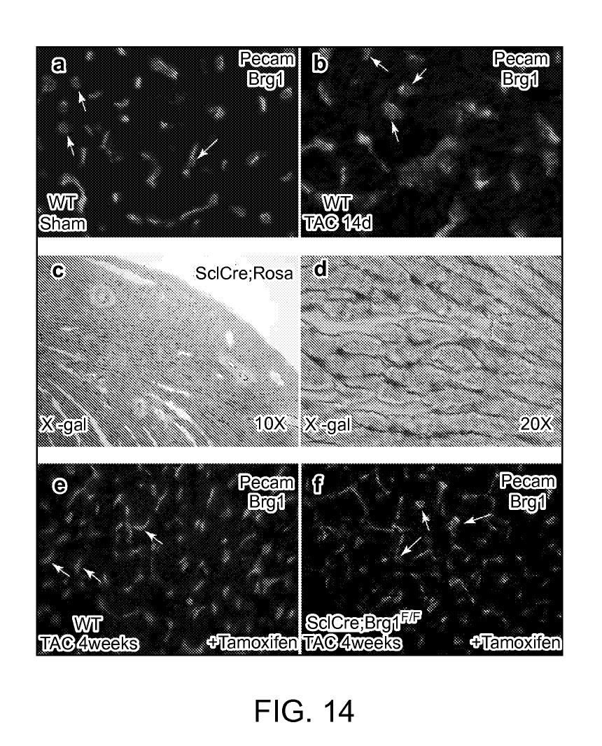

By immunostaining, we observed that Brg1 was expressed at a low level in endothelial cells of normal hearts (FIG. 14a). However, Brg1 level was highly up-regulated in cardiac endothelial cells within 14 days after transaortic constriction (TAC) (FIG. 14b), a procedure that stresses the heart and results in cardiac hypertrophy. Our findings therefore suggest that pressure overloading of the heart by TAC activates the expression of endothelial Brg1.

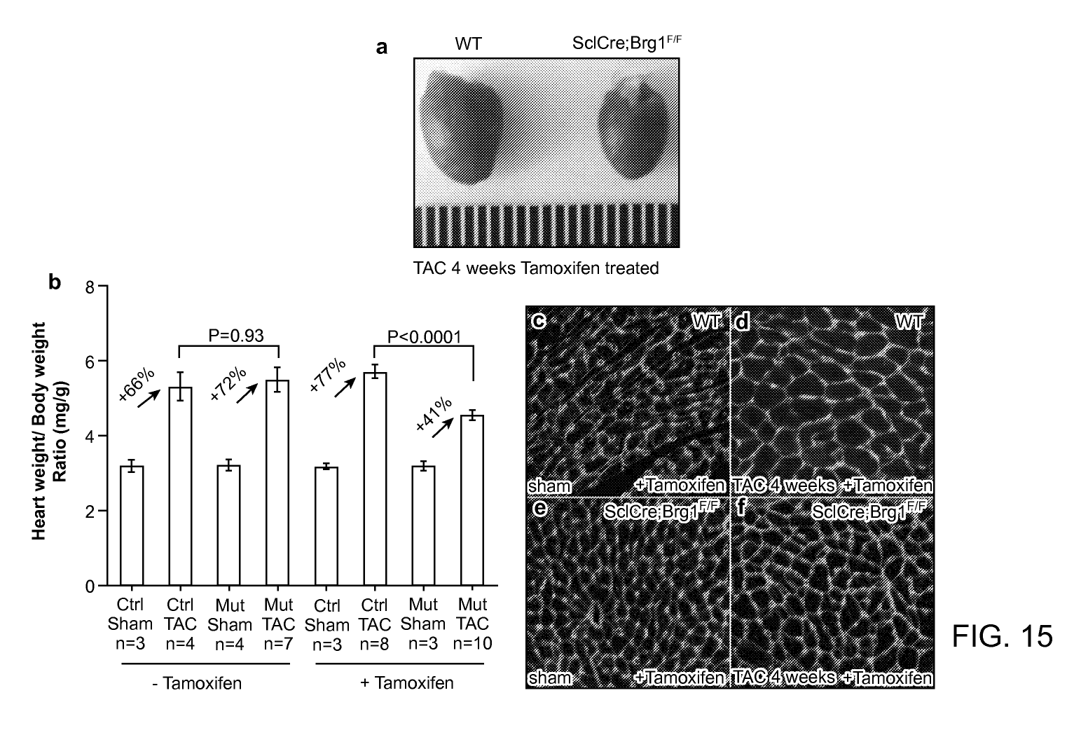

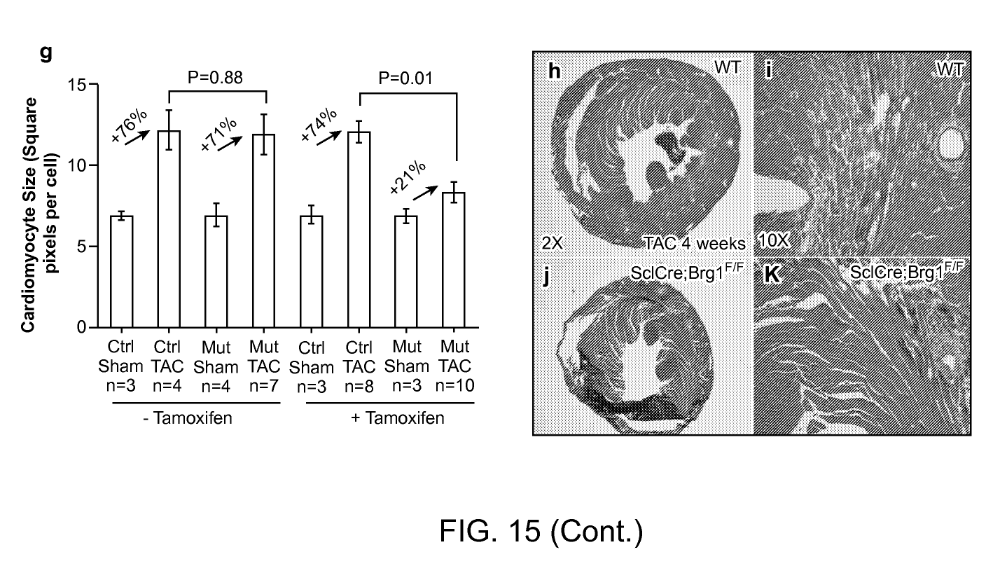

To test whether such endothelial activation of Brg1 is essential for cardiac hypertrophy, we first used a tamoxifen-dependent SclCre^{ER} mouse line to induce endothelial Brg1 deletion in mice that carried floxed alleles of Brg1 gene (Brg1^{F/F}). By immunostaining, we showed that tamoxifen treatment for 5 days before the TAC surgery was sufficient to activate a β-galactosidase reporter (FIG. 14c, 14d) and disrupt endothelial Brg1 activation in stressed hearts (FIG. 14e, 14f). We then performed the TAC procedure to pressure-overload the heart and induce cardiac hypertrophy in control and SclCre^{ER};Brg1^{F/F}littermate mice with or without tamoxifen treatment. Four weeks after TAC, control mice had larger hearts than SclCre^{ER};Brg1^{F/F} mice that lacked endothelial Brg1 (FIG. 15a). Analysis of cardiac mass (ventricular weight/body weight ratio) showed an approximately 50 percent reduction (from 77% to 41%) of cardiac hypertrophy in SclCre^{ER}; Brg1^{F/F} mice that lacked endothelial Brg1 (FIG. 15a, b). Measurement of cardiomyocyte size by wheat germ agglutinin staining (FIG. 15c-f) revealed an approximately 70 percent reduction (from 74% to 21%) of cardiomyocyte size (FIG. 15g) in SclCre^{ER};Brg1^{F/F} mice lacked endothelial Brg1. Also, there was only minimal cardiac fibrosis in mutant SclCre^{ER};Brg1^{F/F} mice compared to control mice (FIG. 2h-k). Overall, Brg1 disruption in endothelial cells reduces cardiac hypertrophy by 50-70% and dramatically reduces cardiac fibrosis. Therefore, endothelial Brg1 is essential for the development of cardiac hypertrophy and cardiomyopathy.

### Clinical Utility:

Brg1 functions in both endothelial cells and cardiomyocytes to promote cardiac hypertrophy, fibrosis, and myopathy. Because endothelial and cardiomyocytic Brg1 act in concert to promote cardiac hypertrophy and myopathy, targeting Brg1 and its associated factors is an effective way of treating patients with cardiomyopathy from various causes.

What is claimed is:

1. A method to suppress cardiac hypertrophy or prevent onset of cardiac hypertrophy in a patient, the method comprising:
administering to said patient an effective amount of an agent that suppresses functional activity or expression of Brg1 or other BAF subunit proteins in cardiomyocytes and/or cardiac endothelial cells.

2. The method according to claim 1, wherein said agent inhibits the expression of Brg1 or other BAF subunit proteins in cardiomyocytes and/or cardiac endothelial cells.

3. The method according to claim 2, wherein said agent is a nucleic acid having a sequence complementary to Brg1 or other BAF subunits genetic sequence

4. The method according to claim 3, wherein said substance is an antisense molecule or RNAi effector.

5. The method according to claim 1, wherein said agent is a small molecule inhibitor.

6. A method for the diagnosis of cardiac hypertrophy in the heart, the method comprising:
determining the differential expression Brg1 or other BAF subunits in cardiomyocytes and/or cardiac endothelial cells.

7. The method according to claim 6, wherein said determining comprises:
contacting a biological sample comprising protein from a patient suspected of suffering from cardiac hypertrophy with an antibody that specifically binds to Brg1 other BAF subunits;
detecting the presence of a complex formed between said antibody and said protein;
wherein increased presence of said complex, compared to a control sample, is indicative of cardiac hypertrophy.

8. The method according to claim 6, wherein said determining step comprises:
contacting a biological sample comprising nucleic acids from a patient suspected of suffering from cardiac hypertrophy with a probe that specifically binds to Brg1 other BAF subunits; detecting the presence of a complex formed between said probe and said nucleic acid;
wherein an increase in the presence of said complex, compared to a control sample, is indicative of cardiac hypertrophy.

9. The method according to claim 8, wherein said biological sample comprises nucleic acids specifically amplified with said sequences.

10. A method for identifying an agent that modulates cardiomyocyte differentiation or proliferation, the method comprising:
combining a candidate biologically active agent with any one of:
(a) a Brg1, other BAF subunit, HDAC or PARP polypeptide;
(b) a cell comprising a nucleic acid encoding and expressing a Brg1, other BAF subunit, HDAC or PARP polypeptide; or
(c) a non-human transgenic animal model for comprising one of: (i) a knockout of a gene corresponding to Brg1, other BAF subunit, HDAC or PARP proteins; (ii) an exogenous and stably transmitted mammalian BAF, HDAC or PARP gene sequence; and
determining the effect of said agent on cardiomyocytes and/or cardiac endothelial cells differentiation or proliferation.

11. The method of claim 10, wherein the effect of said agent is cardiomyocyte or cardiomyocyte progenitor proliferation.

12. The method of claim 10, wherein the effect of said agent is cardiomyocyte or cardiomyocyte expression of myosin heavy chain.

13. The method of claim 10, further comprising determining the activity of said agent in an animal model for cardiac hypertrophy.

14. The method of claim 10, wherein said BAF complex polypeptide is Brg1.

15. The method according to claim 10, wherein said biologically active agent upregulates activity.

16. The method according to claim 10, wherein said biologically active agent inhibits activity.

17. The method according to claim 10, wherein said biologically active agent binds to said polypeptide.

18. A transgenic non-human animal in which Brg1, other BAF subunit, HDAC or PARP genes are selectively deleted in myocardial tissue.

19. The transgenic non-human animal according to claim 18, which is an animal model of cardiac hypertrophy or disease caused thereby.

20. A method of modulating cardiomyocyte progenitor cell differentiation or proliferation, the method comprising:
altering Brg1, HDAC or PARP expression in said cardiomyocyte progenitor, wherein increased expression of Brg1 leads to increased proliferation and expression of β-MHC, or increased expression of HDAC or PARP activity leads to expression of β-MHC, or decreased expression of Brg1, HDAC or PARP activity leads to expression of α-MHC.

21. The method according to claim 20, wherein said cardiomyocyte progenitor cell is maintained in in vitro culture.

22. The method of claim 21, wherein said cardiomyocyte progenitor cells are derived from cultured pluripotent stem cells.


SEQUENCE LISTING (12 sequences)

SEQ ID NO: 1 - DNA, 20 bp, Mouse
acggtgaccataaaggagga

SEQ ID NO: 2 - DNA, 20 bp, Mouse
tgtcctcgatcttgtcgaac

SEQ ID NO: 3 - DNA, 20 bp, Mouse
gccctttgacctcaagaaag

SEQ ID NO: 4 - DNA, 20 bp, Mouse
cttcacagtcaccgtcttgc

SEQ ID NO: 5 - DNA, 19 bp, Mouse
gctggtgaaaaggacctct

SEQ ID NO: 6 - DNA, 20 bp, Mouse
cacaggactagaacacctgc

SEQ ID NO: 7 - DNA, 24 bp, Mouse
cacctaacctcaccaagaagatga

SEQ ID NO: 8 - DNA, 23 bp, Mouse
cttcttgaagtccacaggctttc

SEQ ID NO: 9 - DNA, 20 bp, Homo sapiens
aaaacagatctgcgcttcca

SEQ ID NO: 10 - DNA, 20 bp, Homo sapiens
ttgttacacgtttggcatgg

SEQ ID NO: 11 - DNA, 21 bp, Homo sapiens
agtgctgctgttctgccaaat

SEQ ID NO: 12 - DNA, 20 bp, Homo sapiens
ggctcgttgaaggttttcag